SEQUENCE LISTING (23 sequences)

SEQ ID NO: 1 - DNA, 729 bp, Homo sapiens
  CDS (1)..(729)
atgtctggctgtgatgctggggagggagactgttgttcccggagatgcggcgcgcaggacaaggagcatccaagatacctgatcccagaactttgcaaacagttttaccatttaggctgggtcactgggactggaggaggaattagcttgaagcatggcgatgaaatctacattgctccttcaggagtgcaaaaggaacgaattcagcctgaagacatgtttgtttgtgatataaatgaaaaggacataagtggaccttcgccatcgaagaagctaaaaaaaagccagtgtactcctcttttcatgaatgcttacacaatgagaggagcaggtgcagtgattcatacccactctaaagctgctgtgatggccacacttctctttccaggacgggagtttaaaattacacatcaagagatgataaaaggaataaagaaatgtacttccggagggtattatagatatgatgatatgttagtggtacccattattgagaatacacctgaggagaaaggcctcaaagatagaatggctcatgcaatgaatgaatacccagactcctgtgcagtactggtcagacgtcatggagtatatgtgtggggggaaacatgggagaaggccaaaaccatgtgtgagtgttatgactatttatttgatattgccgtatcaatgaagaaagtaggacttgatccttcacagctcccagttggagaaaatggaattgtgtaa

SEQ ID NO: 2 - protein, 242 aa, Homo sapiens
MSGCDAGEGDCCSRRCGAQDKEHPRYLIPELCKQFYHLGWVTGTGGGISLKHGDEIYIAPSGVQKERIQPEDMFVCDINEKDISGPSPSKKLKKSQCTPLFMNAYTMRGAGAVIHTHSKAAVMATLLFPGREFKITHQEMIKGIKKCTSGGYYRYDDMLVVPIIENTPEEKGLKDRMAHAMNEYPDSCAVLVRRHGVYVWGETWEKAKTMCECYDYLFDIAVSMKKVGLDPSQLPVGENGIV

SEQ ID NO: 3 - DNA, 726 bp, Mus musculus
  CDS (1)..(726)
atgtctggctgtcaagctcaaggagactgttgctcgcggccgtgtggcgcgcaggacaaggagcacccccgattcctgatcccagaactttgcaaacagttttaccatctgggctgggtcaccggcactggagggggaatcagcttgaagcatggcaatgaaatctacattgctccctcaggcgtgcaaaaggaacgcattcagccagaagacatgtttgtgtgtgacattaatgagcaggacataagcgggcctccagcatctaagaagctgaaaaaaagccagtgcactcctcttttcatgaatgcttataccatgagaggagctggcgcagtgattcatacccactctaaagctgctgtgatggctacccttctgtttccaggacaggagtttaaaattacacatcaagagatgatcaaaggaataaggaaatgtacctcaggaggctattacagatacgatgatatgttagtggtacctattattgagaacactcctgaagagaaggatctcaaagaaaggatggctcatgccatgaacgagtacccagactcctgtgcggttcttgtccggcgtcatggggtgtacgtgtggggagaaacatgggagaaagcaaaaaccatgtgtgagtgttatgactacctgtttgacattgctgtctccatgaagaagatgggactcgatccaacacagctcccagttggagaaaatggaattgtgtaa

SEQ ID NO: 4 - protein, 241 aa, Mus musculus
MSGCQAQGDCCSRPCGAQDKEHPRFLIPELCKQFYHLGWVTGTGGGISLKHGNEIYIAPSGVQKERIQPEDMFVCDINEQDISGPPASKKLKKSQCTPLFMNAYTMRGAGAVIHTHSKAAVMATLLFPGQEFKITHQEMIKGIRKCTSGGYYRYDDMLVVPIIENTPEEKDLKERMAHAMNEYPDSCAVLVRRHGVYVWGETWEKAKTMCECYDYLFDIAVSMKKMGLDPTQLPVGENGIV

SEQ ID NO: 5 - protein, 34 aa, Artificial Sequence
  Description of Artificial Sequence  Synthetic Peptide
AAVALLPAVLLALLAPKTLKDRMAHAMNEYPDSC

SEQ ID NO: 6 - protein, 34 aa, Artificial Sequence
  Description of Artificial Sequence  Synthetic Peptide
AAVALLPAVLLALLAPKTLADAMAHTMNEYPDSC

SEQ ID NO: 7 - DNA, 30 bp, Artificial Sequence
  Description of Artificial Sequence  Synthetic Primer
ctaagaagcttgggcaagcctgaggcccag

SEQ ID NO: 8 - DNA, 33 bp, Artificial Sequence
  Description of Artificial Sequence  Synthetic Primer
tagttttagcggccgcgagtctatgtcctgaac

SEQ ID NO: 9 - DNA, 33 bp, Artificial Sequence
  Description of Artificial Sequence  Synthetic Primer
ctaaaaggatccatgtctggctgtgatgctggg

SEQ ID NO: 10 - DNA, 33 bp, Artificial Sequence
  Description of Artificial Sequence  Synthetic Primer
gattttggatccttacacaattccattttctcc

SEQ ID NO: 11 - DNA, 33 bp, Artificial Sequence
  Description of Artificial Sequence  Synthetic Primer
ctaaaaggatccatgtctggctgtcaagctcaa

SEQ ID NO: 12 - DNA, 30 bp, Artificial Sequence
  Description of Artificial Sequence  Synthetic Primer
ctaagtctagactctggctgtgatgctggg

SEQ ID NO: 13 - DNA, 33 bp, Artificial Sequence
  Description of Artificial Sequence  Synthetic Primer
gattttgtcgacttagacaattccattttctcc

SEQ ID NO: 14 - protein, 8 aa, Artificial Sequence
  Description of Artificial Sequence  Synthetic Peptide
KLGTELGS

SEQ ID NO: 15 - DNA, 30 bp, Artificial Sequence
  Description of Artificial Sequence  Synthetic Primer
gattttggatcccacaattccattttctcc

SEQ ID NO: 16 - DNA, 39 bp, Artificial Sequence
  Description of Artificial Sequence  Synthetic Primer
ggaccttcgccatccgcggcgctaaaaaaaagccagtgt

SEQ ID NO: 17 - DNA, 39 bp, Artificial Sequence
  Description of Artificial Sequence  Synthetic Primer
acactggcttttttttagcgccgcggatggcgaaggtcc

SEQ ID NO: 18 - DNA, 45 bp, Artificial Sequence
  Description of Artificial Sequence  Synthetic Primer
cctgaggagaaaggcctcgcagatgccatggctcatacaatgaat

SEQ ID NO: 19 - DNA, 45 bp, Artificial Sequence
  Description of Artificial Sequence  Synthetic Primer
attcattgtatgagccattctatctttgaggcctttctcctcagg

SEQ ID NO: 20 - DNA, 39 bp, Artificial Sequence
  Description of Artificial Sequence  Synthetic Primer
cacatatactccatgagctctgaccagtactgcacagga

SEQ ID NO: 21 - DNA, 39 bp, Artificial Sequence
  Description of Artificial Sequence  Synthetic Primer
tcctgtgcagtactggtcagagctcatggagtatatgtg

SEQ ID NO: 22 - DNA, 32 bp, Artificial Sequence
  Description of Artificial Sequence  Synthetic Primer
cgtggatccccgggatttccggtggtggtggt

SEQ ID NO: 23 - DNA, 26 bp, Artificial Sequence
  Description of Artificial Sequence  Synthetic Primer
gcaggatccgagctcgagtcgactca

(19) United States
(12) Patent Application Publication
(10) Pub. No.: US 2004/0086848 A1
(43) Pub. Date: May 6, 2004

(54) Methods and compositions using polynucleotides and polypeptides of rank-associated inhibitor (rain)

(75) Inventor: Bryant G. Darnay, Houston, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/629,329

(22) Filed: Jul. 29, 2003

Related U.S. Application Data
(60) Provisional application No. 60/399,205, filed on Jul. 29, 2002.

(51) Int. Cl. C12Q 1/70; C07K 14/16

(52) U.S. Cl. 435/5; 530/350

(57) ABSTRACT

The present invention concerns the use of methods to treat bone loss by inhibiting osteoclast precursor cell fusion. Describe herein is the molecular cloning of a polypeptide encoding a protein termed RAIN. The functions of RAIN include regulating osteoclast differentiation and fusion events. In certain embodiments, compositions comprising polynucleotide and/or polypeptides of RAIN may be used in therapeutic compositions and methods for treating bone loss. In various embodiments, bone loss may be treated by inhibiting the differentiation of osteoclast. Inhibition of osteoclast differentiation may be accomplished by inhibiting osteoclast precursor cell fusion, thus inhibiting the formation of a multi-nucleate resorptive osteoclast.

### BACKGROUND OF THE INVENTION

This application claims the benefit of U.S. Provisional Application Serial No. 60/399,205 Entitled “METHODS AN COMPOSITIONS USING POLYNUCLEOTIDES AND POLYPEPTIDES OF RANK-ASSOCIATED INHIBITOR (RAIN),” filed Jul. 29, 2002.

I. Field of the Invention

The present invention relates generally to the fields of Molecular and Cellular Biology. More particularly, it concerns methods and compositions for modulating osteoclastogenesis.

II. Description of Related Art

Living bone tissue is continuously being replenished by the process of resorption and deposition of calcium minerals. This process, described as, the absorption-resorption cycle, is facilitated by two cell types, the osteoblasts and the osteoclasts. The osteoclast is a multinucleated cell and is the only cell in the body known to have the capacity to degrade (or resorb) bone. This resorption activity is accomplished by the osteoclast forming pits (resorption lacunae) in bone tissue. In fact, osteoclast activity in cell culture is measured by their capacity to form these pits on slices of mineralized tissue such as bone or sperm whale dentine. The osteoclast is derived from a hematopoietic precursor which it shares with the formed elements of the blood (Mundy and Roodman, 1987). The precursor for the osteoclast is a mononuclear cell (cell with a single nucleus) which is found in the bone marrow and which forms the mature and unique multinucleated osteoclast after undergoing replication and differentiation by means of cell fusion. The mature osteoclast is distinguished from other multinucleated cells by the presence of the enzyme tartrate-resistant acid phosphatase (TRAP) which is often used as an osteoclast cell marker.

Among the pathological conditions associated with an abnormal osteoclast development or function are conditions wherein increased bone resorption results in the development of fragile and/or brittle bone structure, such as osteoporosis, or increased bone absorption results in the development of excess bone mass, such as osteopetrosis. It is believed that the development of excess or deficient populations of osteoclasts or osteoblasts results from a corresponding lack or excess of specific cytokines in the blood.

Many of the known cytokines stimulate or inhibit blood cells: Several growth regulatory cytokines such as CSF-M, transforming growth factor alpha, interleukin-1 and tumor necrosis factor have been shown to stimulate marrow mononuclear cell proliferation. Although cytokines such as interleukin-1 (IL-1), tumor necrosis factor (TNF) and interleukin-6 (IL-6) may influence osteoclast formation and differentiation (Mundy, 1990), these factors are not specific osteoclast growth regulatory factors.

Knockout mouse models of RANKL, RANK, and osteoprotegerin decoy receptor (OPG) have demonstrated an essential role of these molecules in osteoclastogenesis (i.e., bone remodeling). The biological importance of these molecules is underscored by the induction of severe osteoporosis by targeted disruption of OPG and by the induction of osteopetrosis by targeted disruption of RANKL or by overexpression of OPG (Bucay et al., 1998; Kong et al., 1999; Mizuno et al., 1998). Thus, osteoclast formation may be attributed to the relative ratio of RANKL to OPG in the microenvironment of bone marrow, and alterations in this balance may be a major cause of bone loss in many metabolic bone disorders. Similar to RANKL −/− mice, targeted disruption of RANK also leads to an osteopetrotic phenotype (Dougall et al., 1999; Li et al., 2000). Both RANK −/− and RANKL −/− mice exhibited absence of osteoclasts, indicating the essential requirement of these molecules for osteoclastogenesis. Furthermore, RANK and RANKL are required for lymph node organogenesis and early B and T cell development (Dougall et al., 1999; Kong et al., 1999). Additionally, mice lacking TRAF6 (Lomaga et al., 1999), c-Src (Soriano et al., 1991), c-Fos (Johnson et al., 1992), or the NF-κB subunits p50/p52 (Franzoso et al., 1997; Iotsova et al., 1997) also display an osteopetrotic phenotype; though these mutant mice have osteoclasts, these cells apparently have defects in bone resorption. Thus, RANKL and RANK as well as their cytoplasmic signaling molecules are required for osteoclastogenesis. In vitro, RANKL has been demonstrated to play an essential role in osteoclast differentiation and activation (Hofbauer et al., 2000). Maintenance of bone integrity requires a dynamic balance between bone formation and bone resorption. The net pool size of active osteoclasts is determined by the net effects of differentiation and fusion of osteoclast precursors and by the activity and rate of apoptosis of active osteoclasts. Although various cytokines (TNF, IL-1, IL-6, IL-11, TGFα) and molecules (11α, 25-dihydroxyvitamin D3 and glucocorticoids) expressed by osteoblast lineage cells have been shown to play a role in osteoclast differentiation, it appears that the essential factors are RANKL (produced by osteoblasts) and RANK (expressed on osteoclast progenitors), as well as the resultant intracellular signaling mechanisms and pathways. There is also a requirement for M-CSF, but its function still remains elusive, but is probably only required for the initiation of differentiation of the early osteoclast progenitors and possibly their survival.

Some of the TNF receptor family members have been shown to interact with a family of adaptor proteins known as TRAFs, (TNF receptor-associated factors), which participate in activation of the transcription factor NF-κBand JNK (Arch et al., 1998). The TRAF family consists of six distinct proteins each containing a ring and zinc finger motif in their N-termini and C-terminal domains that appear to be responsible for self-association and protein interaction. The inventor first reported the interaction of TRAF2, TRAF5, and TRAF6 with RANK and demonstrated that RANK could activate both the NF-κB and JNK pathways (Darnay et al., 1998). Subsequently, a more detailed analysis of the interaction of these TRAFs with RANK was reported (Darnay et al., 1999). Also, the inventor identified a novel TRAF6 binding motif in RANK that is distinct from the TRAF2 and TRAF5 binding domains. An identical TRAF6 binding motif in CD40 was described using a combinatorial peptide library approach (Pullen et al., 1999). The TRAF6 binding domain in RANK was sufficient for activation of NF-κB, suggesting that TRAF2 and TRAF5 are not necessary for NF-κB activation. In addition, it appears that the TRAF2 binding motif is sufficient for JNK activation, although the TRAF6 binding domain could also activate JNK, but to a lesser extent.

Additionally, NIK, an NF-κB inducing kinase, was found to also be required for activation of NF-κB by RANK. Collectively, these data support a role for TRAF6 in NF-κB activation and possibly a cooperative role of TRAF2 and TRAF6 in JNK activation by RANK. The inventor's observations were supported by similar reports by others in the field (Galibert et al., 1998; Hsu et al., 1999; Kim et al., 1999; Wong et al., 1998). In addition to TRAF2, TRAF5, and TRAF6, these reports show that TRAF1 and TRAF3 also bind to RANK at its carboxy terminus. In two of these reports, dominant negative TRAF2, TRAF5, and TRAF6 were used to evaluate their role in NF-κB activation by RANK. It appears that all of the dominant negative TRAFs differentially inhibit the activation of NF-κB induced by overexpression of RANK in 293 cells. However, inclusion of all dominant negative TRAFs did not completely eliminate the activation of NF-κB induced by RANK in 293 cells. The role played by each TRAF molecule in RANK signal transduction remains elusive, likely due to the model systems utilized in these experiments. Stimulation of RANK also causes the recruitment of TRAF6, which recruits and activates c-Src, which appears to be responsible for activation of PI3 kinase and AKT (Wong et al., 1999).

### SUMMARY OF THE INVENTION

The invention includes the identification and isolation of a polynucleotide encoding a RANK Associated Inhibitor (RAIN) and polypeptides thereof. The RAIN protein modulates signaling pathways associated with the RANK (a receptor on osteoclast progenitor cells)/RANKL (a ligand for the RANK receptor typically produced by osteoblasts) pathway. RANKL interacts with cell surface RANK, activating signaling adaptor molecules of the TRAF family, which in turn stimulate pathways including NF-κB, PI3 kinase/AKT, JNK, and c-Src. The functions of RAIN include regulating osteoclast differentiation and fusion events. In certain embodiments, compositions comprising polynucleotide and/or polypeptides of RAIN may be used in therapeutic compositions and methods for treating bone loss or bone accretion. In various embodiments, bone loss may be treated by inhibiting the differentiation of osteoclast. Inhibition of osteoclast differentiation may be accomplished by inhibiting osteoclast precursor cell fusion, thus inhibiting the formation of a multi-nucleate resorptive osteoclast.

In various embodiments of the invention, polynucleotides encoding RAIN, RAIN polypeptides or peptides, and agents that increase the activity or expression of RAIN may be used to inhibit signaling through RANK and inhibit osteoclast precursor cell fusion. The inhibition of osteoclast precursor cell fusion inhibits the formation of bone resorptive osteoclasts and thus, decreasing, reducing, slowing or inhibiting the resorption of bone in a subject. The compositions and methods described herein may be used to treat conditions or diseases that result from or are associated with bone resorption. Examples of pathological conditions resulting from or associated with bone resorption include, but are not limited to osteoporosis; cancers, and in particular cancers that have metastasized to the bone; rheumatoid arthritis, osteoarthritis, and the like. Other embodiments of the invention may include the inhibition of the TRAF mediated signaling pathway of a cell. In particular, the TRAF 2, TRAF 5, and/or TRAF 6 mediated pathways. Alternatively, the inhibition of RAIN may increase the osteoclast differentiation and provide modulation of bone accreation, for example osteopetrosis.

Certain embodiments of the invention include an isolated polypeptide comprising at least 10, 15, 20, 25, 30 or more contiguous amino acids of SEQ ID NO: 2, 4, 5 or 6. In various embodiments, a polypeptide may comprise at least 50, 55, 60, 65, 70,75, 80, 90, 95, 100, 110, 120, 130, 140, 150, 160, 170, 180, 190, 200, 210, 220, 230, 240, 241 or lengths therebetween of contiguous amino acids of SEQ ID NO: 2 or 4. In certain embodiments, the use of the full length sequences of SEQ ID NO:2, 4, 5 and 6 are contemplated. The peptide can also comprise a leader sequence operatively coupled to the amino terminus of the peptide or polypeptide. A leader signal sequence can include, but is not limited to a Kaposi fibroblast growth factor signal sequence, HIV-1 Tat (48-60), D-amino acid-substituted HIV-1 Tat (48-60), arginine-substituted HIV-1 Tat (48-60), Drosophila Antennapaedia (43-58), or a polyarginine polypeptide having 6 to 8 arginines.

Embodiments of the invention also include a method of treating a subject with bone loss that includes inhibiting osteoclast precursor cell fusion by administering a polypeptide or peptide comprising at least 10, 15, 20, 25, 30 or any length therebetween of contiguous amino acids of SEQ ID NO: 2, 4, 5 or 6, wherein the polypeptide or peptide modulates RANK signaling. In certain embodiments, the polypeptide can comprise at least 50, 55, 60, 65, 70,75, 80, 90, 95, 100, 110, 120, 130, 140, 150, 160, 170, 180, 190, 200, 210, 220, 230, 240, 241 or lengths therebetween of contiguous amino acids of SEQ ID NO: 2 or 4.

In other embodiments, a method of treating a subject with bone loss can include inhibiting osteoclast precursor cell fusion by administering an effective amount of an expression vector, wherein the expression vector comprises a polynucleotide encoding a RAIN polypeptide under the transcriptional control of a promoter. The promoter can be a constitutive or inducible promoter. The expression vector can be a viral or plasmid vector, as well as an expression cassette. A viral vector can be a vaccinia virus, adenovirus, herpesvirus, retrovirus, cytomegalovirus, or adeno-associated virus. The expression vector may be delivered using a variety of methods known to one of skill in the art and includes, but is not limited to endoscopically, intraveneously, intraarterially, intramuscularly, intralesionally, percutaneously, or subcutaneously delivered vectors, including viral vectors. Administration of a vector or expression cassette may be repeated.

In certain embodiments, osteoclast precursor cell fusion is inhibited by administration of an effective amount of a RAIN polypeptide or peptide. The RAIN polypeptide can have an amino acid sequence as set forth in SEQ ID NO:2, SEQ ID NO:4, SEQ ID NO:5 or SEQ ID NO:6. The RAIN polypeptide can be administered intravascularlly, intraarterially, or intralesionally. In particular embodiments a RAIN polypeptide is administered into a bone cavity. Osteoclast precursor cell fusion can be inhibited by administration of an agent that increases RAIN expression.

Other embodiments include methods of inhibiting TRAF mediated RANK signaling in a cell comprising administering to the cell a RAIN polypeptide as described herein.

Certain embodiments include methods of identifying a modulator of a osteoclast precursor fusion comprising: providing a cell deficient in a RAIN polypeptide; contacting the cell with a candidate substance; and comparing osteoclast cell fusion observed when the candidate substance is not added, wherein an alteration in osteoclast cell fusion indicates that the candidate substance is a modulator of a osteoclast cell fusion. The candidate substance can be a second cell, a cancer cell, a multiple myoloma cell, a peptide, peptide mimetic or small molecule. In particular embodiments, the candidate substance is a small molecule. In certain embodiments, a candidate substance is a RAIN analogue. The RAIN analogue can have agonist or antagonist activity relative to RAIN function or RANK signaling. The cell deficient in a RAIN polypeptide may comprises an inactivated RAIN gene. The cell deficient in a RAIN polypeptide may also express an antisense RAIN nucleic acid.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1D. Molecular cloning of human and mouse RAIN. (FIG. 1A) Yeast cdc25-2 was transformed with expression plasmids encoding the indicated bait and prey proteins and plated on glucose selective medium at 25° C. After 4 days, three individual colonies were picked from each plate and spotted on four plates (two containing glucose and two containing galactose). The plates were incubated at 25° C. or 37° C. as indicated. Subsequent DNA sequencing of clone 61 identified it as the C-terminal region of TRAF2, and clone 72 was identified as a novel cDNA, which the inventor has named RAIN. Arrows indicate growth of clone 61 and 72 on galactose-containing medium at 37° C. Glu, glucose; Gal, galactose. (FIG. 1B) The deduced amino acid sequence of full-length cDNAs of hRAIN and mRAIN were aligned, indicating 95% sequence identity. Those residues that were different between hRAIN and mRAIN were conserved substitutions. (FIG. 1C) A genomic fragment from mouse chromosome 2 of approximately 26 kB derived from the pBAC clone 161K20 (generously provided by Dr. Roe, The University of Oklahoma) is shown. This sequence comprises the 7 exons and 6 introns of the mouse RAIN gene. Of note, the mouse pyruvate dehydrogenase component X (mPDX) gene is located 5′ of exon 1 of RAIN, and its putative transcript runs in the opposite direction. (FIG. 1D) Expression of mouse RAIN by PCR of a cDNA panel (Clontech, mouse panel) containing mouse tissues. PCR was performed using RAIN and GAPDH specific primers and samples were taken at cycles 22, 26, and 30 (indicated by triangle). Sk mus, skeletal muscle; E, embryonic.

FIG. 2A-2C. Interaction of RAIN with endogenous RANK. (FIG. 2A) RAW cells were transfected with pCMV3×FLAG-hRAIN by Fugene according to the manufacturer. After 48 h, cells were selected in G418 (600 μg/ml) for 3 weeks. After an additional week, single colonies were expanded. Expression of FLAG-tagged RAIN in three independent clones was visualized by western blotting with anti-FLAG antibodies (left panel). Cell lysates from RAW cells and RAIN clones were immunoprecipitated with anti-RANK, and co-precipitation of FLAG-tagged RAIN was visualized by western blotting with anti-FLAG antibodies (right panel). The membranes were then stripped and re-probed with the anti-RANK antibody. (FIG. 2B) Parental RAW cells and clones stably expressing RAIN (#1-3) were stimulated with RANKL (10 nM) for the indicated times. Cells were collected, lysates prepared, and proteins (30 μg) were separated by 10% SDS-PAGE and electroblotted onto a nitrocellulose membrane. The membranes were first immunoblotted with phospho-specific ERK antibody and then stripped and re-probed with the anti-ERK antibody. (FIG. 2C) Parental RAW cells and clones stably expressing RAIN (#1-3) were stimulated with RANKL (10 nM) for the indicated times, nuclear extracts were prepared, and gel mobility shift assay for NF-κB was performed. The cytoplasmic extracts (30 μg) were separated by 10% SDS-PAGE and electroblotted onto a nitrocellulose membrane. The membranes were probed with anti-IκBα antibodies.

FIG. 3A-3F. RAW cells stably expressing RAIN do not form multi-nucleated osteoclasts. (FIG. 3A) RAW cells were plated in 24-well plates and incubated with RANKL (30 ng/ml) in the absence or presence of 100 ng/ml of Fc-OPG. Cells were stained for TRAP on day 5 using a TRAP staining kit (Acid Phosphatase Kit, Sigma). (FIG. 3B) Parental RAW cells and RAIN clones (#1-3) were plated in triplicate in 24-well plates and stimulated the following day with RANKL (100 ng/ml). After 5 days the cells were stained for TRAP. Photographs from representative wells of each treatment were taken using a 10× objective lens. No multi-nucleated osteoclasts were observed in any of the RAIN clones examined. (FIG. 3C) Parental RAW cells or RAIN clones (#1-3) were plated in 24 well plates and stimulated the following day with RANKL (100 ng/ml) for 24, 48, and 72 h. Cells were collected, whole cell lysates prepared, and proteins (30 μg) subjected to 10% SDS-PAGE and transferred to nitrocellulose membranes. p27 was visualized by probing with anti-p27 antibody. (FIG. 3D) Parental RAW cells or RAIN clone 3 were plated in 24 well plates and stimulated the following day with RANKL (100 ng/ml) for 24, 48, and 72 h. Cells were collected, whole cell lysates prepared, and proteins (30 μg) subjected to 10% SDS-PAGE and transferred to nitrocellulose membranes. The membranes were probed with an anti-NFATc1 antibody. (FIG. 3E) Human 293 cells were transiently transfected with empty vector or pcDNA3-RAIN. After 24 h, cells were collected and lysed, and lysates (30 μg) were subjected to SDS-PAGE and western blotting with anti-RAIN antibodies (left panel). RAW cells (20×10³/well) were plated in 24-well plates in duplicate and treated with RANKL (50 ng/ml) for the indicated number of days. Whole-cell lysates were prepared and subjected to SDS-PAGE and western blotting with anti-RAIN antibodies (right panel). (FIG. 3F) RAW cells were plated as in (FIG. 3E) and treated with RANKL (100 ng/ml) for the indicated times and total RNA was isolated. Semi-quantitative RT-PCR was performed using specific primers for RAIN, TRAP, mmp9, and actin. Samples were taken after cycles 20, 26, and 30. Shown are the samples taken at cycle 20.

FIG. 4A-4C. Increased osteoclast formation in RAIN anti-sense clones. (FIG. 4A) Parental RAW cells or RAIN anti-sense clones were plated in triplicate in 24-well plates and stimulated the following day with RANKL (10 or 30 ng/ml). After 72 h, the cells were stained for TRAP. Photographs from representative wells of each treatment were taken using a 10× objective lens. The total number of TRAP⁺, multi-nucleated osteoclasts in each well is depicted in the top left corner of each panel. (FIG. 4B) Parental RAW cells (open bars) or RAIN anti-sense clones (AS-1, hatched bars; AS-2, filled bars) were plated in triplicate in 24-well plates and stimulated the following day with RANKL (10, 30, and 100 ng/ml). After 48 h, the cells were stained for TRAP. The total number of TRAP⁺, multi-nucleated osteoclasts were counted. (FIG. 4C) RAW and RAIN-AS2 cells were plated in 6-well plates and transiently transfected with empty vector (2.5 μg), pCMV3×FLAG-hRAIN (0.5, 1.0, and 2.5 μg), or pCR-FLAG-TRAF5 (2.5 μg). After 24 h, the cells were lysed and proteins (30 μg) were subjected to 10% SDS-PAGE and transferred to nitrocellulose membranes. FLAG-tagged RAIN and TRAF5 were visualized by probing with anti-FLAG.

FIG. 5A-5D. Putative actin-binding domain in RAIN. (FIG. 5A) Parental RAW, RAIN clone 3, or RAIN-AS1 cells were plated on cover slips in 24-well plates, and the following day the cells were treated with 100 ng/ml RANKL. After 4 days, the cells were fixed and stained with Alexa Fluor 488 phalloidin. Photographs from representative wells of each treatment were taken using a 40× objective lens using a phase contrast or fluorescent red filter. (FIG. 5B) Following procedures provided by the manufacturer (Cytoskeleton, Inc.), actin was mixed with GST-RAIN (lane 2), α-actinin (lane 3), or bovine serum albumin (BSA, lane 4) and processed as described in Materials and Methods below. Samples were centrifuged, equal volumes of supernatant (S) and pellets (P) were subjected to 10% SDS-PAGE, and proteins were visualized by Coomassie blue staining. Lane 1 is GST-RAIN without the addition of actin (top panel). For the bottom panel, samples were processed as described (lane 1: α-actinin; lane 2: BSA; lane 3: cofilin). (FIG. 5C) Schematic diagram of a sequence alignment modified from Paavilainen et al. (2002) of mouse, human, and yeast twinfilin (1, N-terminal domain; 2, C-terminal domain) and human and yeast cofilin and mouse and human RAIN. Based upon the 3D structure of cofilin and twinfilin, the secondary structures in this region are indicated above the sequence alignment. The critical residues for interaction with actin are indicated by an asterisk. (FIG. 5D) Schematic diagram of human RAIN. Underlined residues in RAIN are the analogous residues in twinfiln and cofilin that were shown to be essential for actin binding (Paavilainen et al., 2002; Paunola et al., 2002). Each of the underlined residues in human RAIN was mutated to the indicated amino acid.

FIG. 6A-6C. Point mutations in RAIN disrupt its inhibitory function. (FIG. 6A) RAW cells stably expressing the indicated RAIN mutation were examined for expression of the mutant RAIN by western blotting with anti-FLAG. (FIG. 6B) Stable RAIN mutant clones were plated in triplicate in 24-well plates and stimulated the following day with RANKL (100 ng/ml). After 4 days the cells were stained for TRAP. Photographs from representative wells were taken using a 10× objective lens; the number in the top left corner indicates the clone number. (FIG. 6C) The indicated clones were plated on synthetic bone discs (Osteologic), treated with RANKL (100 ng/ml), and after 5 days processed as described by the manufacturer. Representative wells were photographed using a 10× objective lens.

FIG. 7. Schematic of RAIN function in osteoclast formation. RANKL interacts with the cell surface receptor RANK and recruits TRAFs (only TRAF2, 5, and 6 are shown) via its cytoplasmic domain. (1) TRAF6 is the mediator of early osteoclast differentiation, which induces activation of the NF-κB, p38, JNK, and ERK pathways. Activation of the early signaling kinases act on transcription factors to regulate genes required for osteoclast commitment, and during this process RAIN is upregulated. (2) The interaction of RAIN with RANK (and possibly TRAF5) at the plasma membrane could localize RAIN to regions where it functions to control actin polymerization. (3) TRAF6 activates the Src pathway for its role in survival and cytoskeleton reorganization during attachment of the osteoclast to the bone forming the sealing zone for bone resorption.

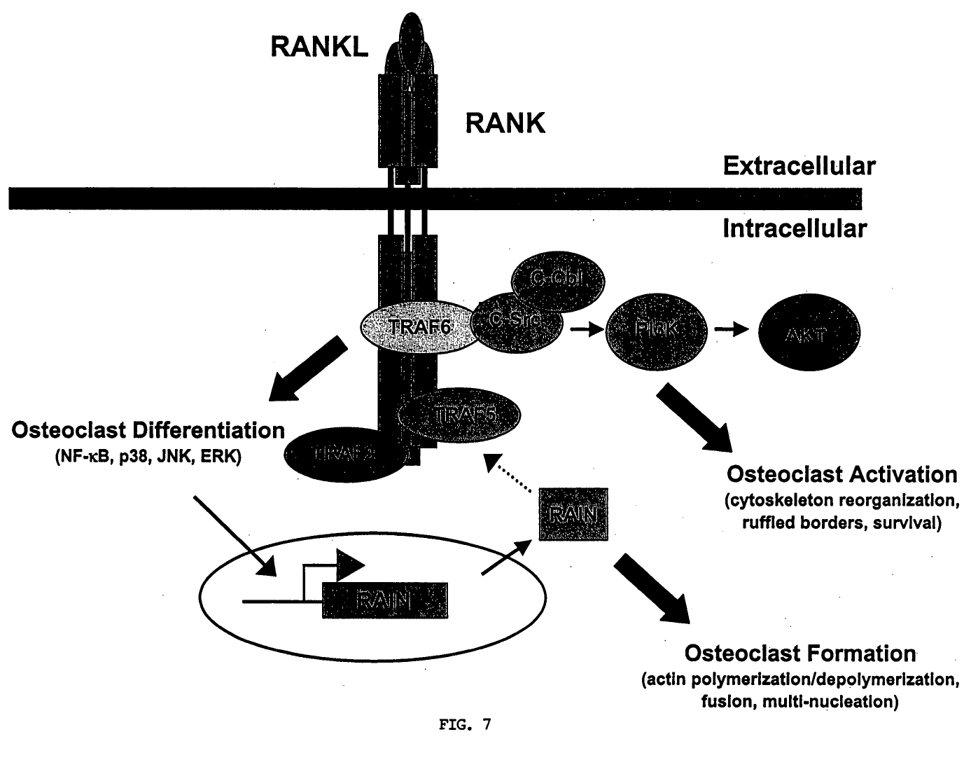

FIG. 8A-8B. Human and Mouse RAIN cDNAs and their deduced amino acid sequence. (FIG. 8A) Full-length mouse RAIN was obtained by PCR screening and aligned with the cDNA of Mmrp19. Shown are the nucleotides that differ in cloned mRAIN as compared to Mmrp19. Each of these differences are silent mutations as indicated by amino acid shown. (FIG. 8B) Full-length human RAIN was obtained by PCR screening and aligned with the cDNA of CGI-29. Shown are the nucleotides that differ in cloned hRAIN, which causes a Thr to Gly changes. The nucleotide difference at the 3′ end is due to the primer used for cloning, but is a silent mutation.

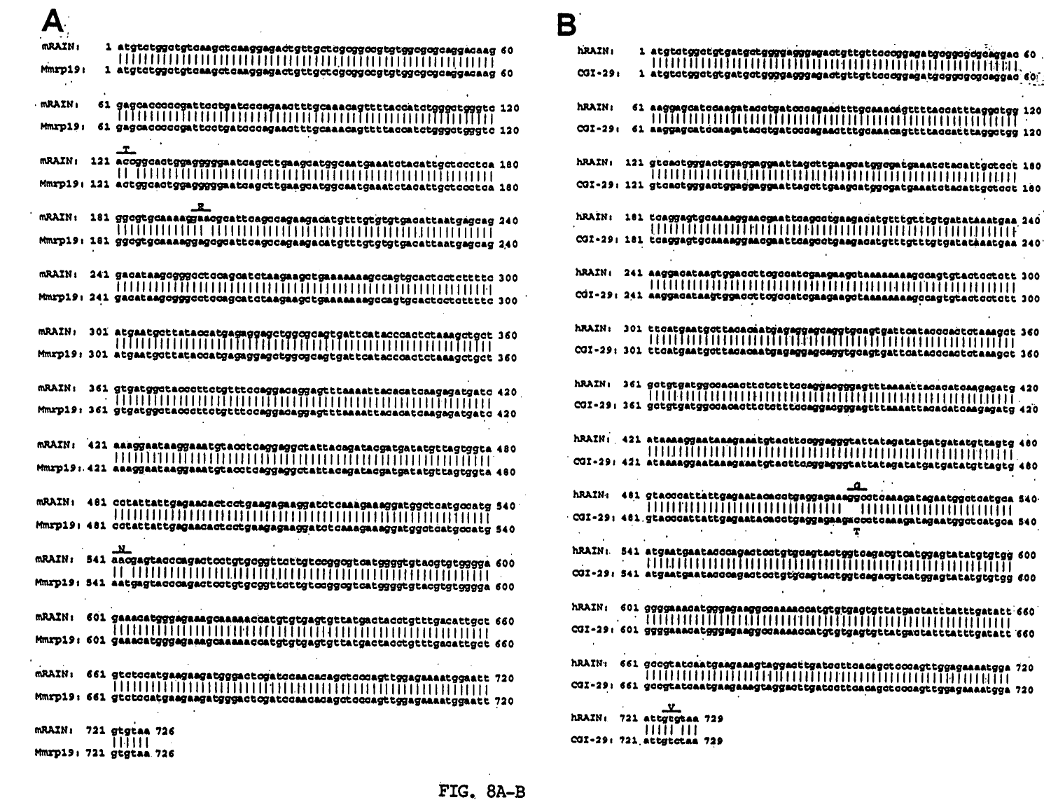

### DESCRIPTION OF ILLUSTRATIVE EMBODIMENTS

The invention includes the identification and isolation of a polynucleotide encoding a RANK associated inhibitor (RAIN) and polypeptides thereof, as well as methods and composition comprising the polynucleotides and poypeptides of the invention. Embodiments of the invention also include methods and compositions for treating or screening for molecules to treat a variety of diseases. The RAIN protein modulates signaling pathways associated with the RANK (a receptor on osteoclast progenitor cells)/RANKL (a ligand for the RANK receptor typically produced by osteoblasts) pathway. RANKL interacts with cell surface RANK activating signaling adaptor molecules of the TRAF family, which in turn stimulate pathways including NF-κB, PI3 kinase/AKT, JNK, and c-Src. Alternatively, a soluble receptor osteoprotegerin decoy receptor (OPG), a member of the TNF receptor family expressed by osteoblasts, may sequester RANKL and inhibit its ability to bind to osteoclast cell surface receptor RANK. It is suggested that the activation of the Src and PI3 kinase pathway may be responsible for cytoskeleton reorganization, while activation of the NF-κB and AKT pathways could be responsible for anti-apoptotic or survival pathways.

Describe herein is the molecular cloning of a polypeptide encoding a protein termed RAIN. The functions of RAIN include regulating osteoclast differentiation and fusion events. In various embodiments of the invention, polynucleotides encoding RAIN, RAIN polypeptides, and agents that increase the activity or expression of RAIN may be used to inhibit osteoclast precursor cell fusion. The inhibition of osteoclast precursor cell fusion inhibits the formation of bone resorptive osteoclasts and thus, decreasing, reducing, slowing or inhibiting the resorption of bone in a subject. The compositions and methods described herein may be used to treat conditions or diseases that result from or are associated with bone resorption. Examples of pathological conditions resulting from or associated with bone resorption include, but are not limited to osteoporosis; cancers, and in particular cancers that have metastasized to the bone; rheumatoid arthritis, osteoarthritis, and the like.

### I. RAIN Nucleic Acids and Polynucleotides

Various embodiments of the invention include nucleic acids encoding a RAIN polypeptide or fragment thereof. The present invention is not limited in scope to these nucleic acids, however, as one of ordinary skill could, using these nucleic acids, readily identify related homologs in these and various other species (e.g., rat, rabbit, dog, monkey, gibbon, human, chimp, ape, baboon, cow, pig, horse, sheep, cat and other species).

In addition, it should be clear that the present invention is not limited to the specific nucleic acids disclosed herein. As discussed below, a “RAIN encoding nucleic acid” may contain a variety of different bases and yet still produce a corresponding polypeptide that is functionally indistinguishable, and in some cases structurally indistinguishable, from RAIN polypeptides disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the activity of RAIN, osteoclast differentiation, osteoclast cell fusion and/or bone formation.

A. Nucleic Acids Encoding RAIN

Nucleic acids according to the present invention may encode an entire RAIN gene, a RAIN polypeptide, a domain of RAIN, or any other fragment of RAIN as set forth herein. The nucleic acid may be derived from genomic DNA, e.g., cloned directly from the genome of a particular organism. In preferred embodiments, however, the nucleic acid comprises a complementary DNA (cDNA). Also contemplated is a cDNA plus a natural intron or an intron derived from another gene; such engineered molecules are sometime referred to as “mini-genes.”

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given RAIN from a given species may be represented by natural variants that have slightly different nucleic acid sequences but, nonetheless, encode the same protein (see Table 1 below).

As used in this application, the term “a nucleic acid encoding a RAIN” refers to a nucleic acid molecule that has been isolated free of total cellular nucleic acid. In preferred embodiments, the invention concerns a nucleic acid sequence as set forth in SEQ ID NO: 1 or SEQ ID NO:3. The term “as set forth in SEQ ID NO: 1 or SEQ ID NO:3” means that the nucleic acid sequence substantially corresponds to a portion of SEQ ID NO:1 or SEQ ID NO:3. The term “functionally equivalent codon” is used herein to refer to codons that encode the same amino acid, such as the six codons for arginine or serine (Table 1, below), and also refers to codons that encode biologically equivalent amino acids, as discussed herein.

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO:1 or SEQ ID NO:3 are contemplated. Sequences that are essentially the same as those set forth in SEQ ID NO:1 or SEQ ID NO:3 may also be functionally defined as sequences that are capable of hybridizing to a nucleic acid segment or the complement of SEQ ID NO:1 or SEQ ID NO:3 under standard conditions.

The DNA segments of the present invention include those encoding functionally equivalent RAIN proteins and peptides, as described herein. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein or peptide structure may be engineered, based on considerations of the properties of the amino acids being exchanged. Changes designed by man may be introduced through the application of site-directed mutagenesis techniques or may be introduced randomly and screened later for the desired function, as described below.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

B. Oligonucleotide Probes and Primers

The present invention also encompasses DNA segments that are complementary, or essentially complementary, to the sequence set forth in SEQ ID NO:1 or SEQ ID NO:3. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 or SEQ ID NO:3 under relatively stringent conditions such as those described herein. Such sequences may encode entire RAIN proteins, or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Such oligonucleotides will find use, for example, as probes in Southern and Northern blots and as primers in amplification reactions. In preferred embodiments, the oligonucleotides will hybridize specifically to the nucleic acid sequence of SEQ ID NO:1 or SEQ ID NO:3. Longer polynucleotides encoding 250, 500, 1000, 1200, 1500, 2000, 2500, 3000 or 5000 bases and longer are contemplated as well.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to RAIN or, more particularly, homologs of RAIN from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex. Typically, a primer of about 17 to 25 nucleotides in length is preferred. In general, site-directed mutagenesis, as well as other mutagenesis techniques, is described in Sambrook et al. 2001, which is incorporated herein by reference.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Modulators of RAIN Expression

Various methods for modulating the expression of a gene or protein encoded by a gene are known to one of ordinary skill in the art, see Current Protocols in Molecular Biology, Chapter 26 “Gene Silencing” or similar texts and papers.

Although proteins traditionally have been used for catalysis of nucleic acids, another class of macromolecules has emerged as useful in this endeavor. Ribozymes are RNA complexes that cleave nucleic acids in a site-specific fashion (Kim and Cook, 1987; Gerlach et al., 1987; Forster and Symons, 1987; Michel and Westhof, 1990; Reinhold-Hurek and Shub, 1992; Joyce, 1989). For an example see U.S. Pat. No. 5,354,855, which is incorporated herein by reference. Another method of targeting a particular molecule that is expressed at an undesired time or level is the intracellular production of an antibody or antibody fragment (intrabody) which is capable of binding to a specific target, see U.S. Pat. No. 6,004,940, which is incorporated herein by reference.

The methods of the present invention may select and use nucleic acids that bind to and modulate characteristics of RAIN. Thus, in certain embodiments, a nucleic acid, may comprise or encode an aptamer. An “aptamer” as used herein refers to a nucleic acid that binds a target molecule through interactions or conformations other than those of nucleic acid annealing/hybridization described herein. Methods for making and modifying aptamers, and assaying the binding of an aptamer to a target molecule may be assayed or screened for by any mechanism known to those of skill in the art (see for example, U.S. Pat. Nos. 5,840,867, 5,792,613, 5,780,610, 5,756,291 and 5,582,981, which are incorporated herein by reference).

Still further methods that may be used to modulate expression of RAIN include antisense methodology. For exemplary antisense methodology see U.S. Pat. Nos. 6,238,921, 5,734,039, at least.

### II. RAIN Peptides and Polypeptides

The RAIN polypeptide is a protein translated from the RAIN mRNA, which is produced by the RAIN gene. In addition to an entire RAIN molecule, the present invention also relates to fragments of the polypeptides that may or may not retain various functions described herein. Fragments, including the N-terminus of the molecule, may be generated by genetic engineering of translation stop sites within the coding region (discussed below). Alternatively, treatment of the RAIN polypeptide with proteolytic enzymes, known as proteases, can produce a variety of N-terminal, C-terminal and internal fragments. Examples of fragments may include contiguous residues of SEQ ID NO:2, SEQ ID NO:4, SEQ ID NO:5 or SEQ ID NO:6 of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35,36, 37, 38, 39, 40, 41, 42, 43, 44, 45, 50, 55, 60, 65, 75, 80, 85, 90, 95, 100, 200, 300 or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration). These fragments may also be synthesized or produced by recombinant methods known in the art.

A. Variants of RAIN

Amino acid sequence variants of the polypeptide or peptides of RAIN can be substitutional, insertional or deletion variants. Deletion variants lack one or more residues of the native protein or peptide that are not essential for function or immunogenic activity. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate or interacting molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity. Table 1 (above) shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still obtain a biologically equivalent and immunologically equivalent protein.

Another embodiment for the preparation of polypeptides according to the invention is the use of peptide mimetics. Mimetics are peptide-containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of RAIN, but with altered and even improved characteristics, as well agonist or antagonistic properties.

B. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals, transmembrane regions or leader sequences.

In particular embodiments, RAIN peptides are fused to leader sequence to provide a RAIN peptide that has an improve cellular uptake. A leader sequence/RAIN peptide fusion can be used to provide an agonistic or antagonistic molecule for the modulation of RAIN activity. For example, the present invention may utilize cell penetrating peptides in the methods and compositions described herein. In particular a cell penetrating peptide may be fused to the terminus of a peptide of the present invention. The cell penetrating peptide facilitates intracellular delivery of the desired peptide into a target cell, such as a osteoclast or a progentitor to an osteoclast. The technology of protein transduction is known in the art, such as is described in Rojas et al. (1998) and Schwarze et al. (1999), both incorporated by reference herein.

Several cell-penetrating peptides (CPP) have been identified from proteins including TAT from human immunodeficiency virus (HIV) 18 19, and fibroblast growth factor (Ludewig et al., 1999; Frankel and Pabo, 1988). The CPPs have the ability to deliver peptides or proteins in a variety of cell types, in vitro and in vivo. For a more detailed description of cell pentrating peptides see for example U.S. Provisional Patent application 20030077289, which is incorporated herein by reference.

C. Purification of Proteins

In certain embodiments, it may be desirable to purify RAIN, fragments or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis, and isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein” or “purified peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

D. Synthetic Peptides

The present invention also describes smaller RAIN-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

E. Antigen Compositions

The present invention also provides for the use of RAIN proteins or peptides as antigens for the immunization of animals relating to the production of antibodies. It is envisioned that RAIN, or portions thereof, will be coupled, bonded, bound, conjugated or chemically-linked to one or more agents via linkers, polylinkers or derivatized amino acids. This may be performed such that a bispecific or multivalent composition, antibody or vaccine is produced. It is further envisioned that the methods used in the preparation of these compositions will be familiar to those of skill in the art and should be suitable for administration to animals and/or humans, i.e., pharmaceutically acceptable. Preferred agents are the carriers are keyhole limpet hemocyannin (KLH) or bovine serum albumin (BSA).

### III. Osteoclasts

The human skeleton is continuously remodeled, normally turning over in about 2 years and allows use of skeletal mineral in calcium homeostasis. The strength and shape of the skeleton is preserved by segmental replacement: a bone section is degraded by osteoclasts, formed from monocyte-macrophage precursors (Scheven et al., 1986; and Fujikawa et al., 1996), while osteoblasts, derived from stromal cells, synthesize new bone (Rickard, et al., 1996). These unrelated cells differentiate in a coupled manner, producing a new bone section in a few weeks.

Osteoclasts, which are multinucleated giant cells, arise from hematopoietic stem cells and are the primary cells responsible for physiological and pathological bone resorption. Osteoclasts are specialized for the removal of both the inorganic and organic phases of bone (Blair et al., 1986). Changes in the levels of cytokines and growth factors in bone microenvironment cause abnormal bone resorption by the osteoclasts (for a review see Mundy, et al., 1997). Accordingly, forced expression of IL-4 (Lewis, et al., 1993), and G-CSF (Takahashi, et al., 1996) in mice induced osteopenia, while mice overexpressing soluble TNF-α receptor (Ammann, et al., 1997) or depleted of the IL-6 gene (Poli, et al., 1994) are protected against bone loss caused by estrogen deficiency.

Dissolution of the hydroxyapatite mineral phase is dependent upon acidification of the subosteoclastic resorption lacuna, via the action of carbonic anhydrase II and a proton pump (Vaes, 1968; Baron et al., 1985; Blair and Schlesinger, 1992).

Excessive bone resorption by osteoclasts contributes to the pathology of many human diseases including arthritis, osteoporosis, periodontitis, and hypercalcemia of malignancy. During resorption, osteoclasts remove both the mineral and organic components of bone (Blair et al., 1986).

The current major bone diseases of public concern are osteoporosis, hypercalcemia of malignancy, osteopenia due to bone metastases, periodontal disease, hyperparathyroidism, periarticular erosions in rheumatoid arthritis, Paget's disease, immobilization-induced osteopenia, and glucocorticoid treatment.

All these conditions are characterized by bone loss, resulting from an imbalance between bone resorption (breakdown) and bone formation, which continues throughout life at the rate of about 14% per year on the average. However, the rate of bone turnover differs from site to site, for example it is higher in the trabecular bone of the vertebrae and the alveolar bone in the jaws than in the cortices of the long bones. The potential for bone loss is directly related to turnover and can amount to over 5% per year in vertebrae immediately following menopause, a condition which leads to increased fracture risk. Turnover may be effected by an increase or decrease in osteoblast activity or an increase or decrease in osteoclast activity. Compositions and methods for modulating osteoclast and/or osteoblast in a subject would be useful in the treatment of a variety of diseases or conditions associated with bone loss.

All the conditions listed above would benefit from treatment with agents which inhibit bone resorption. One mechanism for the inhibition of bone resorption is the inhibition of osteoclast precursor cell fusion.

### IV. Vectors for Cloning, Gene Transfer and Expression

Within certain embodiments expression vectors are employed to express a RAIN polypeptide product, which can then be purified and, for example, be used to vaccinate animals to generate antisera or monoclonal antibody with which further studies may be conducted or to modulate the activity or RAIN or a polynucleotide encoding such. In other embodiments, the expression vectors are used in gene therapy. Expression requires that appropriate signals be provided in the vectors, and which include various regulatory elements, such as enhancers/promoters from viral and/or mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

Below is a list of viral promoters, cellular promoters/enhancers and inducible promoters/enhancers that could be used in combination with the nucleic acid encoding a gene of interest in an expression construct (Table 2 and Table 3). Additionally, any other promoter/enhancer combination (for example, as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin | Banerji et al., 1983; Gilles et al., 1983; Grosschedl |
| Heavy Chain | et al., 1985; Atchinson et al., 1986, 1987; Imler et |
| | al., 1987; Weinberger et al., 1984; Kiledjian et al., |
| | 1988; Porton et al.; 1990 |
| Immunoglobulin | Queen et al., 1983; Picard et al., 1984 |
| Light Chain | |
| T-Cell Receptor | Luria et al., 1987; Winoto et al., 1989; Redondo et |
| | al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-Dra | |
| β-Actin | Kawamoto et al., 1988; Ng et al.; 1989 |
| Muscle Creatine | Jaynes et al., 1988; Horlick et al., 1989; Johnson et |
| Kinase (MCK) | al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsh et al., 1990 |
| Adhesion Molecule | |
| (NCAM) | |
| α₁-Antitrypain | Latimer et al., 1990 |
| H2B (TH2B) | Hwang et al., 1990 |
| Histone | |
| Mouse and/or Type | Ripe et al., 1989 |
| I Collagen | |
| Glucose-Regulated | Chang et al., 1989 |
| Proteins (GRP94 | |
| and GRP78) | |
| Rat Growth | Larsen et al., 1986 |
| Hormone | |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987; Glue |
| | et al., 1988 |
| Hepatitis B Virus | Bulla et al., 1986; Jameel et al., 1986; Shaul et al., |
| | 1987; Spandau et al., 1988; Vannice et al., 1988 |
| Human | Muesing et al., 1987; Hauber et al., 1988; |
| Immunodeficiency | Jakobovits et al., 1988; Feng et al., 1988; Takebe |
| Virus | et al., 1988; Rosen et al., 1988; Berkhout et al., |
| | 1989; Laspia et al., 1989; Sharp et al., 1989; |
| | Braddock et al., 1989 |
| Cytomegalovirus | Weber et al., 1984; Boshart et al., 1985; Foecking |
| (CMV) | et al., 1986 |
| Gibbon Ape | Holbrook et al., 1987; Quinn et al., 1989 |
| Leukemia Virus | |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., |
| | | 1987b; McNeall et al., |
| | | 1989 |
| MMTV (mouse | Glucocorticoids | Huang et al., 1991; Lee |
| mammary tumor virus) | | et al., 1981; Majors et |
| | | al., 1983; Chandler et al., |
| | | 1983; Lee et al., 1984; |
| | | Ponta et al., 1985; Sakai |
| | | et al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large T | Taylor et al., 1989, |
| | Antigen | 1990a, 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

In certain embodiments of the invention, a cell may contain a nucleic acid construct of the present invention. A cell may be selected or identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. The selectable marker employed is not believed to be important, so long as it is capable of being expressed simultaneously with the nucleic acid encoding a gene product.

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). By virtue of the IRES element, each open reading frame is accessible to ribosomes for efficient translation. Multiple genes can be efficiently expressed using a single promoter/enhancer to transcribe a single message.

There are a number of ways in which expression constructs may be introduced into cells. In certain embodiments of the invention, a vector (also referred to herein as a gene delivery vector) is employed to deliver the expression construct. By way of illustration, in some embodiments, the vector comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Where viral vectors are employed to deliver the gene or genes of interest, it is generally preferred that they be replication-defective, for example as known to those of skill in the art and as described further herein.

One of the preferred methods for in vivo delivery of expression constructs involves the use of an adenovirus expression vector. “Adenovirus expression vector” is meant to include those constructs containing adenovirus sequences sufficient to (a) support packaging of the construct and (b) to express a polynucleotide that has been cloned therein. In this context, expression does not require that the gene product be synthesized. Other than the requirement that the adenovirus vector be replication defective, or at least conditionally defective, the nature of the adenovirus vector is not believed to be crucial to the successful practice of the invention. The adenovirus may be selected from any of the 42 different known serotypes or subgroups A-F. Adenovirus type 5 of subgroup C is a preferred starting material for obtaining a replication-defective adenovirus vector for use in the present invention. This is, in part, because Adenovirus type 5 is a human adenovirus about which a great deal of biochemical and genetic information is known, and it has historically been used for most constructions employing adenovirus as a vector. Adenovirus vectors have been used in eukaryotic gene expression (Levrero et al., 1991; Gomez-Foix et al., 1992) and vaccine development (Grunhaus and Horwitz, 1992; Graham and Prevec, 1992).

Retroviruses may also be used as gene delivery vectors. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively.

Viral vectors that may be employed as expression constructs in the present invention include vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. In general, viral vectors accomplish delivery of the expression construct by infecting the target cells of interest. Alternatively to incorporating the expression construct into the genome of a viral vector, the expression construct may be encapsidated in the infectious particle.

Several non-viral gene delivery vectors for the transfer of expression constructs into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, nonspecific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression vector may simply consist of naked recombinant DNA or plasmids comprising the expression construct. Transfer of the construct may be performed by any of the methods mentioned above which physically or chemically permeabilize the cell membrane.

In still another embodiment of the invention, transferring of a naked DNA expression construct into cells may involve particle bombardment. This method depends on the ability to accelerate DNA-coated microprojectiles to a high velocity allowing them to pierce cell membranes and enter cells without killing them (Klein et al., 1987).

In a further embodiment of the invention, the expression construct may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### V. Antibodies Reactive With a RAIN Polypeptide

Various embodiments of the invention (e.g., therapeutic, diagnostic, screening and prognostic methods) will utilize compositions that preferentially or specifically identify RAIN proteins, polypeptides or peptides. The present invention contemplates an antibody that is immunoreactive with a RAIN molecule of the present invention, or any portion thereof. An antibody can be a polyclonal or a monoclonal antibody. Means for preparing and characterizing antibodies are well known in the art (see, e.g., Harlow and Lane, 1988, incorporated herein by reference).

Briefly, a polyclonal antibody is prepared by immunizing an animal with an immunogen comprising one or more polypeptide of the present invention and collecting antisera from that immunized animal. A wide range of animal species can be used for the production of antisera. Typically an animal used for production of anti-antisera is a nonhuman animal including rabbits, mice, rats, hamsters, pigs or horses. Because of the relatively large blood volume of rabbits, a rabbit is a preferred choice for production of polyclonal antibodies.

Monoclonal antibodies of the present invention will find useful application in standard immunochemical procedures, such as ELISA and Western blot methods and in immunohistochemical procedures such as tissue staining, as well as in other procedures which may utilize antibodies specific to RAIN-related epitopes. Additionally, it is proposed that monoclonal antibodies specific to the particular RAIN of different species may be utilized in other useful applications.

In general, both polyclonal and monoclonal antibodies against RAIN may be employed in antibody cloning protocols to obtain cDNAs or genes encoding other RAIN. They may also be used in inhibition studies to analyze the effects of RAIN related peptides in cells or animals. RAIN antibodies will also be useful in immunolocalization studies to analyze the distribution of RAIN during various cellular events, for example, to determine the cellular or tissue-specific distribution of RAIN polypeptides under different points in the cell cycle. A particularly useful application of such antibodies is in purifying native or recombinant RAIN, for example, using an antibody affinity column. The operation of all such immunological techniques will be known to those of skill in the art in light of the present disclosure.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

MAbs may be readily prepared through the use of well-known techniques, such as those exemplified in U.S. Pat. No. 4,196,265, incorporated herein by reference. Typically, this technique involves immunizing a suitable animal with a selected immunogen composition, (e.g., a purified or partially purified RAIN protein, polypeptide or peptide or cell expressing high levels of RAIN). The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

### VI. Detecting, Diagnosing and Treating Aberrant Expression of RAIN

RAIN may play an important role in the development of osteoclast and in diseases related to an increase or decrease in bone resorption. For example, RAIN may be involved in the molecular mechanisms of diseases related to or associated with bone resorption. Thus, in another embodiment, there are provided methods for diagnosing defects in RAIN expression and function. More specifically, regulatory perturbations relating to RAIN, as well as increases or decreases in levels of expression, may be assessed using standard technologies. Disease may include, but is not limited to osteoporosis, osteoarthritis, as well as cancer of the breast, uterus, ovary, kidney, testis, prostate, skin, intestine, stomach, liver and other tissues, particularly cancers that have metastasized to the bone and other diseases described herein.

A. Genetic Diagnosis

One embodiment of the instant invention comprises a method for detecting variation in the expression of RAIN. This may comprise determining the level of RAIN or determining specific alterations in the expressed product.

A suitable biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow, bone, kidney or cells or tissue derived from these tissues or organs. Other embodiments include fluid samples such as peripheral blood, lymph fluid, ascites, serous fluid, pleural effusion, sputum, cerebrospinal fluid, lacrimal fluid, stool or urine.

Nucleic acids can be isolated from cells contained in the biological sample, according to standard methodologies (Sambrook et al., 2001). The nucleic acid may be genomic DNA, or fractionated or whole cell RNA. Where RNA is used, it may be desired to convert the RNA to a complementary DNA. In one embodiment, the RNA is whole cell RNA; in another, it is poly-A RNA. The nucleic acid may also be amplified using known nucleic amplification procedures.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects may be identified by the present methods. Thus, “alterations” should be read as including overexpression, deletions, insertions, point mutations and duplications. Point mutations result in stop codons, frameshift mutations or amino acid substitutions. Somatic mutations are those occurring in non-germline tissues. Mutations in and outside the coding region also may affect the amount of RAIN produced, both by altering the transcription of the gene or in destabilizing, stabilizing or otherwise altering the processing of either the transcript (mRNA) or protein.

It is contemplated that other mutations in the RAIN genes may be identified in accordance with the present invention. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™ SSCP.

1. Primers and Probes

The term primer, as defined herein, is meant to encompass any nucleic acid that is capable of priming the synthesis of a nascent nucleic acid producing RAIN in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, I⁴C, ³⁵S, ³H, or other label), with a fluorophore (rhodamine, fluorescein) or a chemillumiscent (luciferase).

2. Template Dependent Amplification Methods

A number of template dependent processes are available to amplify sequences present in a given template sample to identify the presence of the RAIN protein. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

A reverse transcriptase PCR™ amplification procedure may be performed in order to quantify the amount of mRNA amplified. Methods of reverse transcribing RNA into cDNA are well known and described in Sambrook et al., 2001. Alternative methods for reverse transcription utilize thermostable, RNA-dependent DNA polymerases. These methods are described in WO 90/07641. Polymerase chain reaction methodologies are well known in the art.

Another method for amplification is the ligase chain reaction (“LCR”), disclosed in EPO patent 320,308, incorporated herein by reference in its entirety. U.S. Pat. No. 4,883,750 describes a method similar to LCR for binding probe pairs to a target sequence. Other methods based on ligation of two (or more) oligonucleotides in the presence of nucleic acid having the sequence of the resulting “di-oligonucleotide”, thereby amplifying the di-oligonucleotide, may also be used in the amplification step of the present invention, Wu and Wallace (1989), incorporated herein by reference in its entirety.

3. Southern/RNA Blotting

Blotting techniques are well known to those of skill in the art. Southern blotting involves the use of DNA as a target, whereas RNA blotting (northern blotting) involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

4. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 2001.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

5. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols, see Sambrook et al., 2001. For example, chromophore or radiolabel probes or primers identify the target during or following amplification.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the RAIN genes that may then be analyzed by direct sequencing.

6. Kit Components

All the essential materials and reagents required for detecting and sequencing RAIN and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

B. Immunologic Diagnosis

Antibodies of the present invention can be used in characterizing the RAIN content of healthy and diseased tissues, through techniques such as ELISAs, immunohistochemisry and Western blotting. These methods may provide a screen for the presence or absence of osteoclast or metastatic cancer, as well as a predictor of disease prognosis.

The use of antibodies of the present invention in an ELISA assay is contemplated. For example, anti-RAIN antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting the same to a second antibody having specificity for RAIN that differs from the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween®, or borate buffer.

The antibody compositions of the present invention will find great use in immunoblot, ELISA or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

In other embodiments, cell based diagnostics may be used. That is a cell lacking or with a reduced level of RAIN may be used to contact a cancer cell for the purpose of determine the propensity of the cell to induce or initiate osteoclastogenesis. The induction of osteoclast precursor fusion is indicative of the involvment or possible involvment of the tested cancer cell in the development of osteolytic lesions or pathologic bone resorption.

### VII. RAIN as a Therapeutic

The certain embodiments the present invention includes the treatment of disease states related to the modulation of expression and/or function of RAIN. In particular, it is envisioned that RAIN activity plays a role in bone homeostasis and resorption. Thus, decreasing levels of RAIN, or reducing or eliminating the activity of RAIN, is believed to provide therapeutic intervention in certain bone loss conditions and cancers.

There also may be situations where one would want to inhibit RAIN function or activity, for example, where overexpression or unregulated expression had resulted in an increase of bone deposition, osteopetrosis. In this case, one would take steps to interfere with or block the expression or function of RAIN. In certain embodiments, inhibition or reduction in the expression of RAIN may used to provide a cell line in which osteoclast precursor cell fusion occurs much more rapidly. These cells may be used in improved screening methods to identify an agent that inhibits osteoclast precursor cell fusion and thus is a candidate for a therapeutic agent for osteoporosis and other diseases associated with bone loss.

In certain embodiments, RAIN peptides, polypeptides and/or variants thereof amy be used as a therapeutic. For example, isolated polypeptide of 10, 11, 12, 13, 14, 15, 16, 17, 18, 19, 20, 25, 30, 40, 50 or more amino acids, including intervening lengths of amino acid are contemplated. In particular embodiments RAIN peptides comprising the amino acid sequences (171-188 WT) AAVALLPAVLLALLAPKTLKDRMAHAMNEYPDSC (SEQ ID NO:5) or RAIN peptides comprising the amino acid sequence (171-188 MUT) AAVALLPAVLLALLAPKTLADAMAHTMNEYPDSC (SEQ ID NO:6) are contemplated. Any RAIN peptide may be fused or operatively coupled to a leader sequence for proper localization in a cell or an organism of interest.

A. Genetic Based Therapies

One of the therapeutic embodiments contemplated by the present inventor is the intervention, at the molecular level, in the events involved in bone loss and in some cancers. Specifically, the present inventors intend to provide, to a osteoclast precursor cell, a modulator capable of increasing, decreasing, activating or inhibiting RAIN in that cell. In certain embodiments, the positive modulator of RAIN may be an expression construct providing a RAIN protein. In certain other embodiments, a negative regulator of RAIN, such as an anti-sense nucleic acid, may be provided. The lengthy discussion of expression vectors and the genetic elements, as well as anti-sense methodology employed therein is incorporated into this section by reference. Particularly preferred expression or anti-sense expression vectors are viral vectors such as adenovirus, adeno-associated virus, herpesvirus, vaccinia virus and retrovirus. Also preferred are liposomally-encapsulated expression vectors.

Those of skill in the art are aware of how to apply gene delivery to in vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below. Various routes are contemplated, including local and systemic, but targeted provision to an osteoclast precursor cell or a cancer cell of interest is preferred.

B. Combined Therapy

In many clinical situations, it is advisable to use a combination of distinct therapies. Thus, it is envisioned that, in addition to the therapies described above, one would also wish to provide to the patient more “standard” therapies. Examples of standard therapies include drugs, radiation, gene therapy, or chemotherapeutics.

Combinations may be achieved by contacting osteoclast precursor cells or cancer cells with a single composition or pharmacological formulation that includes both agents, e.g., a RAIN modulating agent and another therapy for treating aberrant bone metabolism or cancer, or by contacting the cell with two distinct compositions or formulations, at the same time, wherein one composition includes the RAIN modulating therapy and the other includes another treatment agent, such as a chemotherapy. Alternatively, RAIN modulating may precede or follow the other treatment by intervals ranging from minutes to weeks. In embodiments where the other agent(s) are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hr of each other and, more preferably, within about 6-12 hours of each other, with a delay time of only about 12 hr being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a positive or negative modulator of RAIN nucleic acid or protein, or the other agent will be desired. Various combinations may be employed, where a negative or positive modulator of RAIN is “A” and the other therapeutic agent, such as modulator of Traf or a chemotherapeutic is “B”, as exemplified below:

1 A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated as well.

C. Formulations and Routes for Administration to Patients

Where clinical applications are contemplated, it will be necessary to prepare pharmaceutical compositions—expression vectors, virus stocks, aptamers, peptides and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors, peptides and the like stable and allow for uptake by target cells. Buffers also will be employed when recombinant cells are introduced into a patient. Aqueous compositions of the present invention comprise an effective amount of the RAIN modulator to cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, or topical. Alternatively, administration may be by orthotopic, intradermal, subcutaneous, intramuscular, intraperitoneal, intravascular or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dose could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VIII. RAIN Expressing Cell Lines

A particular embodiment of the present invention provides host cells that contain RAIN-related constructs. Host cells expressing RAIN or fragment thereof, including recombinant cell lines derived from such host cells, may be useful in methods for screening for and identifying agents that modulate a function or activity of RAIN, and thereby alleviate pathology related to the over- or under-expression of these molecules or in particular diagnose a propensity to for involvment in formation of osteolytic lesions. The use of constitutively expressed RAIN provides a model for over-regulated expression and the use of knockout, or targeted disruption as well as an antisense expressing cell line provides a model for over under-regulated expression.

In a general aspect, a host cell is produced by the integration of a RAIN transgene into the genome in a manner that permits the expression or targeted disruption of the RAIN gene or mRNA.

### IX. Screening Assays

The present invention also contemplates the screening of compounds for various abilities to interact and/or affect RAIN expression or function, as well as the ability to induce or inhibit osteoclast precursor cell fusion. Particularly preferred compounds will be those useful in inhibiting osteoclast precursor cell fusion in tissues or organs. In the screening assays of the present invention, the candidate substance may first be screened for basic biochemical activity—e.g., binding to RAIN—and then tested for its ability to modulate activity or expression, at the cellular, tissue or whole animal level.

A. Assay Formats

The present invention provides methods of screening for modulators of osteoclast precursor cell fusion. In one embodiment, the present invention is directed to a method of:

(i) providing a cell that exhibits an increase rate of cell fusion;

(ii) contacting the cell with a candidate substance(s); and

(iii) determining the effect of the candidate substance on the ability of the cell to undergo cell fusion.

In still yet other embodiments, one would look at the effect of a candidate substance on the activity of RAIN or other molecules identified as affecting osteoclast precursor cell fusion. This may involve looking at any of a number of cellular characteristics, including steroid hormone responsive genes or expression of a reporter gene.

A candidate substance may include a second cell. A second cell may be derived from a patient having or suspected of having a disease. In particular embodiments the disease may be cancer or another disease with a related osteolytic (bone destroying or resorptive) character. For example, a multiple myoloma may be tested for its ability to induce or otherwise initiate osteoclast progentor cell fusion, which will be indicative of the ability of the cell to cause or contribute to osteolytic lesions. Use of a second cell may be used to diagnose and identify important characteristics of a cell.

Assays may be performed in vitro or in vivo (e.g., in mammalian, yeast, or bacterial cells). Expression constructs may contain all or part of the polynucleotides involved in binding or expression of the reporter construct. Fusion proteins DNA binding or transactivation domains may also be incorporated, such as those used in the yeast two hybrid screens.

B. Inhibitors and Activators

An inhibitor according to the present invention may be one which exerts an inhibitory effect on the ability of a cell line to undergo cell fusion or osteoclastogeneiss. By the same token, an activator according to the present invention may be one which exerts a stimulatory effect on the ability of a cell line to undergo cell fusion.

C. Candidate Substances

As used herein, the term “candidate substance” refers to any molecule that may potentially modulate osteoclast precursor cells or other cells. The candidate substance may be a protein or fragment thereof, a small molecule inhibitor, or even a nucleic acid molecule. It may prove to be the case that the most useful pharmacological compounds will be compounds that are structurally related to compounds which interact naturally with the cellular mechanisms of cell fusion. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like a RAIN, and then design a molecule for its ability to interact with or mimic the functions of RAIN. Alternatively, one could design a partially functional fragment of a RAIN (binding but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of a steroid hormone receptor repressor.

Other suitable inhibitors include antisense molecules, ribozymes, aptamers, intrabodies, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

D. In vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a RAIN molecule or fragment thereof is provided.

The target may be either free in solution, fixed to a support, expressed in or on the surface of a cell. Either the target or the compound may be labeled, thereby permitting determining of binding. In another embodiment, the assay may measure the inhibition of binding of a target to a natural or artificial substrate or binding partner (such as a RAIN). Competitive binding assays can be performed in which one of the agents (RAIN for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, RAIN and washed. Bound polypeptide is detected by various methods.

Purified target, such as a RAIN, can be coated directly onto plates for use in the aforementioned drug screening techniques. However, non-neutralizing antibodies to the polypeptide can be used to immobilize the polypeptide to a solid phase.

E. In cyto Assays

Various cell lines that express or do not express RAIN can be utilized for screening of candidate substances. For example, cells containing a RAIN with engineered indicators can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (cell fusion, reporter gene expression, etc.). Alternatively, molecular analysis may be performed in which the function of a RAIN and related pathways may be explored. This involves assays such as those for protein expression, enzyme function, substrate utilization, mRNA expression (including differential display of whole cell or polyA RNA) and others.

F. Production of Inhibitors or Activators

In an extension of any of the previously described screening assays, the present invention also provide for method of producing inhibitors or activators. The methods comprising any of the preceding screening steps followed by an additional step of “producing the candidate substance identified as a negative or positive modulator of” the screened activity (cell fusion, or osteoclastogenesis).

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Materials and Methods

Reagents, Cell Lines, and Antibodies

The mouse macrophage cell line RAW264.7 (referred to as RAW) and human embryonic kidney 293 cells were obtained from the American Type Culture Collection (Rockville, Md.). RAW264.7 and 293 cells were cultured in DMEM-F12 and MEM, respectively, supplemented with 10% fetal bovine serum and antibiotics. Monoclonal antibodies to phospho-ERK, p38, and JNK were purchased from New England Biolabs, goat anti-rabbit IgG-conjugated horseradish peroxidase from BioRad Laboratories; rabbit polyclonal antibodies against JNK1, IκBα, Myc, TRAF2, TRAF5, and TRAF6 and monoclonal antibody for NFATc1 from Santa Cruz Biotechnology; and goat anti-mouse IgG-conjugated horseradish peroxidase from Transduction Laboratories. Monoclonal antibody to HA was a generous gift from Dr. G. B. Mills (University of Texas M. D. Anderson Cancer Center). Protein A/G Sepharose beads and the Seize primary immunoprecipitation kit were purchased from Pierce. Monoclonal anti-FLAG, glutathione-agarose, and a tartrate resistance acid phosphatase (TRAP) kit were purchased from Sigma. We generated rabbit polyclonal antibodies against two peptides in RAIN (residues 60-72 and 162-175) based upon antigenicity and surface exposure plots. The rabbit antiserum was affinity purified against a bacterial-expressed GST-RAIN fusion protein. The RAIN antibody cross-reacted with GST-RAIN, but not GST.

Yeast Two-Hybrid Screen

RRS screening in yeast was conducted essentially as described (Broder et al., 1998) with some modifications. All reagents were generous gifts from Dr. A. Aronheim (Technion-Israel Institute of Technology). Plasmids pADNS-Ras(61)-RANKcd and pGAP were transfected into host yeast strain cdc25-2 and selected on yeast synthetic medium containing 2% glucose (SD-glucose) lacking tryptophan and leucine. Approximately 5 colonies from this plate were grown in 2×1 ml SD-glucose -Leu, -Trp dropout medium with shaking at 25° C. overnight. The next morning the culture was transferred to 2×250 ml YPD medium and grown at 25° C. with shaking for 5 hr. The cultures were centrifuged and the cells were made competent by the lithium-acetate/TE method. The cells were transformed with 100 μg of a human thymus library in the vector backbone pMYR (Stratagene) and plated on 20×150 mm plates containing SD-galactose -Leu, -Trp, -Ura dropout medium with a layer of glucose on each plate (300 μl of 7% glucose). The plates were incubated at 25° C. for approximately 3 days until small colonies appear and then shifted to 37° C. for up to 14 days. Large colonies were picked, suspended in 50 μl of SD-glucose medium, and spotted on three plates using a grid: two SD-glucose (-Leu, -Trp, -Ura) and one SD-galactose (-Leu, -Trp, -Ura). One of the SD-glucose plates was placed at 25° C. (i.e., master plate) and the other two plates were placed at 37° C. for 2-4 days until colonies appeared. To eliminate any false positives, this plating was repeated a second time. Those colonies that demonstrated galactose-dependent growth at 37° C. were considered positive, and yeast plasmid DNA isolation was performed using lyticase and a modified miniprep protocol. The pMYR library plasmids were transformed into DH5α and selected for chloramphenicol resistance. Miniprep DNA was isolated from the clones, and plasmids were subjected to restriction analysis and DNA sequencing.

Plasmids

The expression vector of full-length murine RANKL (also known as TNF-related activation-induced cytokine (TRANCE) (pcDNA3.1-TRANCE)) was generously provided by Y. Choi (Rockefeller University, New York, N.Y.). To generate a bacterial expression vector for RANKL, we used forward primer (BGD-094) 5′-CTAAGAAGCTTGGGCAAGCCTGAGGCCCAG-3′ (SEQ ID NO:7) and reverse primer (BGD-095) 5′-TAGTTTTAGCGGCCGCGAGTCTATGTCCTGAAC-3′ (SEQ ID NO:8) with HindIII and NotI sites, respectively, to amplify the DNA, which encodes residues 157-316 of mRANKL from the pcDNA3.1-TRANCE template. The PCR product was digested with HindIII/NotI and ligated in-frame with an HA-tag (N-terminal) and a histidine tag (C-terminal) into the expression vector pHB6 (Boerhinger Mannheim). The soluble RANKL was expressed and purified using Ni-affinity chromatography essentially as described for purification of GST-fusion proteins(Darnay et al., 1999). Expression vectors and purification of GST-Jun(1-79) have been previously described (Darnay et al., 1998; Darnay et al., 1999). E expression plasmids encoding FLAG-TRAF2, FLAG-TRAF5, Flag-TRAF6, and myc-TRAF2 have been previously described (Darnay et al., 1999). The N-terminus of TRAF5 (residues 1-233) and TRAF6 (residues 1-269) were constructed by PCR using specific primers and cloned into the EcoRI and BamHI site of pCMV3×FLAG-14.

To obtain the full-length human and mouse RAIN, primers were designed based on the sequences deposited in the database (human CGI-29 and mouse Mmrp19, accession numbers AF132963 and AB028863, respectively, each of which is incorporated herein by reference). PCR was performed with a human thymus plasmid library and the forward primer (BGD-111) 5′-CTAAAAGGATCCATGTCTGGCTGTG ATGCTGGG-3′ (SEQ ID NO:9) and the reverse primer (BGD-113) 5′-GATTTTGGATCCTTACACAATTCCATTTTCTCC-3′ (SEQ ID NO:10) to amplify human RAIN. PCR was performed with a mouse mixed-tissue plasmid library (generously provided by Dr. G. Lozano, University of Texas M. D. Anderson Cancer Center) as the template with the forward primer (BGD-112) 5′-CTAAAAGGATCCATGTCTGGCTGTCAAGCTCAA-3′ (SEQ ID NO: 11) and the reverse primer (BGD-113) to amplify mouse RAIN. The PCR products were digested with BamH I and ligated into pcDNA3 and verified by DNA sequencing. The plasmid pGEX-KG-RAIN was constructed using pcDNA3-hRAIN as the template in a PCR reaction with the forward primer (BGD-114) 5′-CTAAGTCTAGACTCTGGCTGTG ATGCTGGG-3′ (SEQ ID NO:12) and the reverse primer (BGD-115) 5′-GATTTTGTCGACTTAGACAATTCCATTTTCTCC-3′ (SEQ ID NO:13). The PCR product was digested with XbaI and SalI and ligated into pGEX-KG. Plasmid pCMV-FLAG2-RAIN was constructed by inserting a HindIII/XbaI fragment from pcDNA3-hRAIN directly into pCMV-FLAG2, which created an N-terminal FLAG-tag and an additional 8 residues (KLGTELGS (SEQ ID NO:14)) on the N-terminus of hRAIN. Plasmid pCMV3×FLAG-hRAIN was constructed by PCR amplification of hRAIN from pcDNA3-hRAIN using the forward primer (BGD-111) and the reverse primer (BGD-128) 5′-GATTTTGGATCCCACAATTCCATTTTCTCC-3′ (SEQ ID NO:15) and ligating the BamHI-digested fragment into pCMV3×FLAG-14 (Sigma), which results in a 3×FLAG tag at the C-terminus of RAIN. Human RAIN was cloned into the BamHI site of pcDNA3.1-HA (a generous gift from Dr. K. Fujisi) with an HA-tag at the N-terminus.

Site-specific mutations of RAIN were generated by the QuikChange site-directed mutagenesis kit (Stratagene). For each mutant, the template pCMV3×FLAG-hRAIN was used with the following forward and reverse primers creating unique restriction sites: K90/91A (SacI) (5′-GGACCTTCGCCATCCGCGGCGCTAAAAAAAGCCAGTGT-3′ (SEQ ID NO:16) and 5′-ACACTGGCTTTTTTTTAGCGCCGCGGATGGCGAA GGTCC-3′ (SEQ ID NO:17)); K174A/R176A/A180T (NcoI) (5′-CCTGAGGAGAAAGGCCTCGCAGATGCCATGGCTCATACAATGAAT-3′ (SEQ ID NO: 18) and 5′-ATTCATTGTATGAGCCATTCTATCTTTGAGGCCTTTC TCCTCAGG-3′ (SEQ ID NO:19)); and R194A (SacI) 5′-CACATATACTCCATGAGCTCTGACCAGTACTGCACAGGA-3′ (SEQ ID NO:20) and 5′-TCCTGTGCAGTACTGGTCAGAGCTCATGGAGTATATGTG-3′ (SEQ ID NO:21)). Each of the mutations was verified by DNA sequencing.

The plasmid template pGEX-KG-RANKcd has been previously described(Darnay et al., 1999). PCR was performed using pGEX-KG-RANKcd as the template and forward primer 5′-CGTGGATCCCCGGGATTTCCGGTGGTGGTGGT-3′ (SEQ ID NO:22) and reverse primer 5′-GCAGGATCCGAGCTCGAGTCGACTCA-3′ (SEQ ID NO:23). The PCR product was digested with BamHI and ligated into pYesRas(61)deltaF-BamHI. This vector was checked for correct orientation by SmaI digest which resulted in pYesRas(61)deltaF-RANKcd. This vector was digested with HindIII/NotI, and the isolated fragment was ligated into pADNS digested with HindIII/NotI, resulting in the plasmid pADNS-Ras(61)-RANKcd, and its identity was confirmed by DNA sequencing.

Generation of RAW Cells Stably Expressing FLAG-Tagged RAIN

RAW cells were plated at 0.5×10⁶ cells/well on 6-well plates and transfected the next day with pCMV3×FLAG-hRAIN (wild type or mutants) or pcDNA3-mRAIN-antisense (2.5 μg total DNA) using 9 μl of Fugene (Boerhinger Mannheim). After 48 h, cells were trypsinized and plated in 100 mm dishes in the presence of G418 (600 μg/ml). Single colonies were isolated after 2 weeks of G418 selection, expanded, and examined for expression of RAIN by western blotting with anti-FLAG antibodies. All RAIN clones were derived from the same stock of parental RAW cells.

Osteoclast Differentiation and Synthetic Bone Resorption Assay

RAW cells or RAIN clones were cultured in 24 dishes at a density of 1×10⁴ cells/well, and then treated with RANKL (10-100 ng/ml). Typically on day 4 or 5, cells were fixed and stained for TRAP and in some instances counter-stained with hematoxylin. Osteoclast formation was assessed by counting the total number of multi-nucleated (>3 nuclei), TRAP-positive cells present per well in triplicate. Alternatively, RAW cells or RAIN clones were plated in 24-well plates containing synthetic bone discs (Osteologic) and the following day treated with RANKL (100 ng/ml). After 5 days , the synthetic bone discs were processed as described by the manufacturer. Photographs were taken using a 10× objective lens.

F-Actin Staining

RAW cells or RAIN clones were cultured on cover slips in 24-well dishes at a density of 1×10⁴ cells/well, and treated the following day with RANKL (100 ng/ml). After 4 days, the cells on the cover slips were fixed with 3.7% formaldehyde at room temperature for 15 min. The cover slips were washed 3 times with PBS and then permeabilized with acetone on ice for 5 min. The cells were rehydrated in PBS for 10 min, washed 3 times with PBS, then blocked with 1% BSA for 20 min. The cells were stained with Alexa Fluor 488 phalloidin diluted 1:40 (Molecular Probes) for 20 min, washed 3 times with PBS, washed 3 times with water, and then dried for mounting.

Electrophoretic Mobility Shift Assay

Nuclear extracts were prepared from transfected cells essentially as described (Haridas et al., 1998). Equivalent amounts of nuclear protein were used in an EMSA reaction with ³²P-labeled NF-κ B oligonucleotide from the HIV-LTR as described (Haridas et al., 1998).

Semi-ouantitative RT-PCR

RAW cells were plated in 24-well plates and treated with RANKL (100 ng/ml) for 12, 24, 36, 48, and 72 h (three plates per time point). At the indicated times, RAW cells were harvested, and total RNA was extracted using Triazol reagent. RT-PCR was performed with a set of specific primers for RAIN, TRAP, mmp9, and actin as indicated by the manufacturer using a Superscript RT-PCR kit (Invitrogen). Samples were taken after cycles 20, 26, and 30 and evaluated by agarose gel electrophoresis.

Actin Co-sedimentation Assays

F-actin co-sedimentation assays were performed essentially as described by the manufacturer (Cytoskeleton, Inc.). Briefly, nonmuscle actin was resuspended to a final concentration of 1 μg/gl in general actin buffer (5 mM Tris-HCl pH 8.0, 0.2 mM CaCl₂, 0.2 mM ATP) and allowed to sit on ice. After 60 min, 40-μg aliquots of actin were mixed with the indicated test proteins in a final volume of 50 μl for 30 min at room temperature followed by the addition of actin polymerization buffer (final concentration: 5 mM Tris-HCl pH 8.0, 0.2 mM CaCl₂, 25 mM KCl, 0.5 mM MgCl₂, 0.7 mM ATP) for an additional 30 min at room temperature. Samples were then centrifuged in a 50Ti rotor at 40,000 rpm for 1.5 h at room temperature. The supernatants were removed and the pellets dissolved SDS-sample buffer (same volume as the supernatants) and equal volumes of supernatants and pellets were subjected to 10 or 13% SDS-PAGE, and the proteins visualized by staining the gels with Coomassie blue.

Western Blot Analysis and Immunoprecipitations

Lysates were prepared from RAW cells stimulated with RANKL as indicated in the figure legends. Approximately 30 μg of protein was subjected to 10% SDS-PAGE, transferred to nitrocellulose membranes, probed with the appropriate antibodies, and developed using the enhanced chemiluminescence (ECL) system (Amersham). For JNK kinases assays, approximately 30 μg of lysate from treated cells was used for immunoprecipitation with anti-JNK1 and protein A/G sepharose beads for 1 h. Beads were collected by centrifugation, washed three times in lysis buffer, and then washed two times in low-salt buffer. JNK activity was analyzed using exogenous GST-Jun(1-79) as a substrate as previously described (Darnay et al., 1999).

Antibodies against RANK, FLAG, TRAF2, TRAF5, and TRAF6 were crosslinked to beads using the Seize primary immunoprecipitation kit. Lysates from either 293 or RAW cells programmed to express epitope-tagged proteins were mixed with the appropriate antibody-beads in lysis buffer (20 mM TRIS, pH 7.4, 250 mM NaCl, 1 mM DTT, 1 mM sodium orthovanadate, 2 mM EDTA, 1% Triton X-100, 2 μg/ml leupeptin, and 2 μg/ml aprotinin) and rotated overnight at 4° C. The beads were collected by centrifugation, washed three times in lysis buffer, and then washed once in low-salt buffer (20 mM TRIS, pH 7.4, 50 mM NaCl, and 1 mM DTT). Bound proteins were eluted with addition of SDS-sample buffer and boiled. The eluted proteins were subjected to SDS-PAGE, and western blot analysis was performed with the appropriate antibodies and developed using ECL.

### Example 2

Identification of RAIN by its Interaction With the Cytoplasmic Domain of RANK

The inventor sought to identify signaling molecules that are associated with the intracellular domain of RANK and used the recently developed yeast two-hybrid system called the ras recruitment system (RRS) (Broder et al., 1998). This system is based on the ability of a mutant RAS fusion protein to be localized to the plasma membrane through the interaction between two hybrid proteins. Ras membrane localization complements a yeast temperature-sensitive mutant in the Ras guanyl nucleotide exchange factor, Cdc25-2. This yeast strain grows at 25° C. but cannot grow at 37° C.

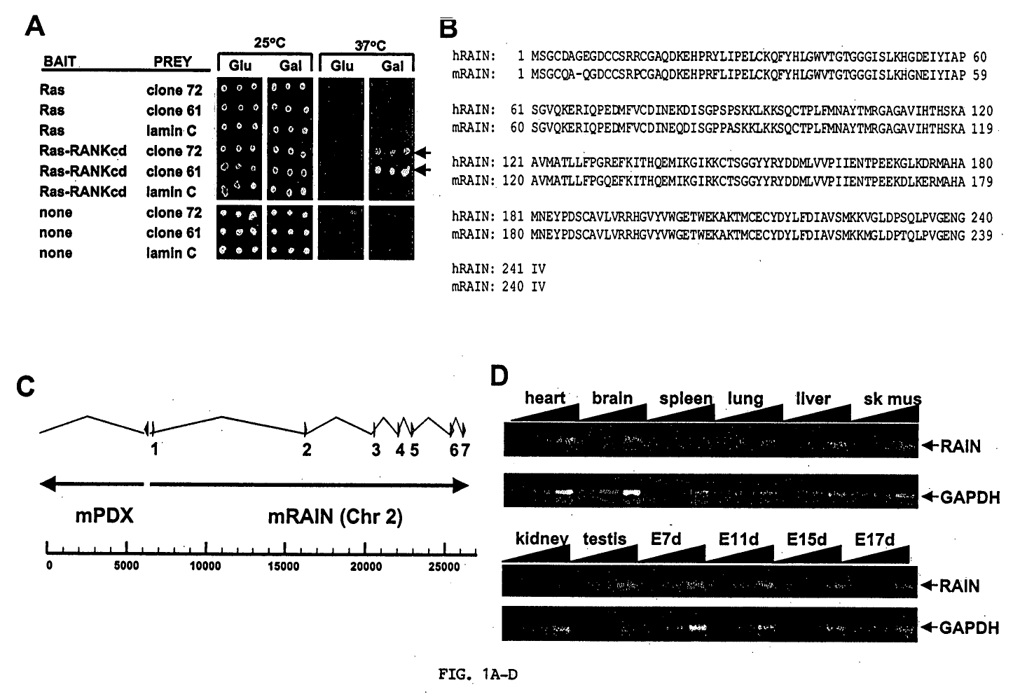

Ahuman thymus library was screened using the RANK cytoplasmic domain fused to the C-terminus of Ras (Ras-RANKcd). Of the 13 putative clones identified in this screen, five clones (i.e., clone 61) encoded the C-terminal region of TRAF2, a protein known to interact with RANK (Darnay et al., 1999), which confirmed that this system is effective in identifying RANK-interacting molecules. One other clone (i.e., clone 72) specifically interacted with RANK as indicated by galactose-dependent growth at 37° C. (FIG. 1A ). All transformants grew on glucose or galactose at 25° C. with the appropriate amino acid selection markers, indicating successful transformation of the expression plasmids. Since no growth occurred on galactose at 37° C., clones 61 and 72 did not interact with Ras nor did they complement cdc25-2 when expressed alone. However, when the RAS-RANKcd fusion protein was expressed, both clones 61 and 72 exhibited galactose-dependent growth at 37° C., indicating that the proteins expressed from pMYR-61 and -72 interacted with RANKcd (FIG. 1A). As a negative control, lamin C did not interact with Ras or Ras-RANKcd. These data identified clone 72 as a protein that interacts with the cytoplasmic domain of RANK in yeast.

### Example 3

Molecular Cloning of RAIN

Based upon a search of the Genebank database, clone 72 appeared to contain a novel sequence with no assigned function; the sequence was identical to human CGI-29 and murine homologue Mmrp19. To obtain the full-length human and mouse cDNA sequences of clone 72, cDNA were amplified by PCR of a human thymus and mouse mixed-tissue library and cloned the cDNA into pcDNA3. The complete nucleotide sequence of the cDNA predicted an open reading of 242 and 241 amino acids for the human and mouse sequences, respectively (FIG. 1B), which were 95% identical. Based on functional data as described below, this protein was named RAIN, for RANK-Associated Inhibitor of osteoclast differentiation.

Extensive searches of the available public databases did not reveal any proteins that were homologous to RAIN. Additionally, RAIN does not possess any known domains or motifs from which the function of RAIN might be inferred. Various NCBI analysis programs indicated that RAIN does not contain a signal sequence, transmembrane domain, nuclear localization signal, or endoplasmic reticulum retention signal; thus RAIN is most likely a cytoplasmic protein, which was further confirmed by cell fractionation (data not shown). Additionally, RAIN was shown to form homodimers as judged by yeast two-hybrid analysis and migration during gel filtration chromatography (data not shown).

Based on the human genome project, the human gene for RAIN was mapped to chromosome 11p12-13 and contains 7 exons covering 34 kB. Similar to hRAIN, the genomic organization of mouse RAIN contained 7 exons spanning 20 kB, and was located on chromosome 2 (FIG. 1C). Expression of mouse RAIN was examined by PCR of a cDNA panel of mouse tissues (FIG. 1D). RAIN was primarily expressed in all tissues with lower levels expressed in spleen and kidney. RAIN was also expressed during embryogenesis with little variation between embryonic day 7 through 17 (FIG. 1D). Furthermore, EST database searches revealed the presence of numerous EST sequences that were similar to RAIN and were expressed in prostate, senescent fibroblasts, colon, fetus, endothelial cell, germinal B cell, adrenal gland, Jurkat T cell, aorta, CNS, heart, kidney, tonsil, uterus, whole embryo, thymus, and tongue.

### Example 4

RAIN Interacts With RANK in RAW264.7 Cells

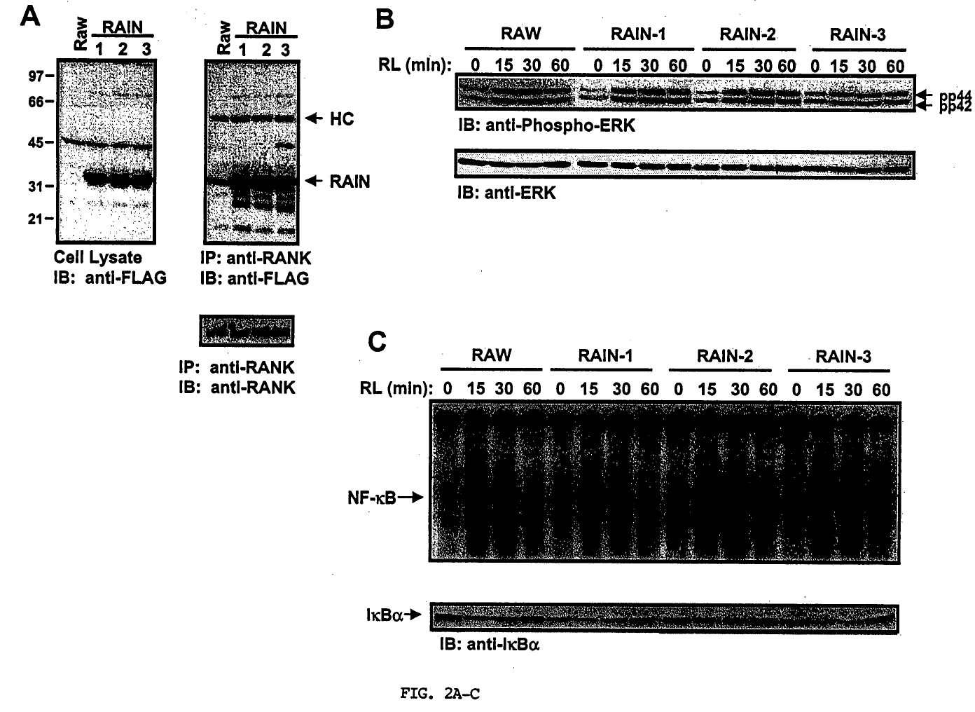

To verify the interaction of RAIN with RANK, RAW264.7 cells (referred to hereafter as RAW) were used, which express endogenous RANK. RAW cells serve as a model for RANKL-induced signal transduction and osteoclast differentiation (Collin-Osdoby et al., 2003; Ishida et al., 2002; Ye et al., 2002). First, RAW clones were established that stably expressed FLAG-tagged RAIN (i.e., RAIN clones #1-3) as indicated by western blotting with anti-FLAG (FIG. 2A, left panel). The interaction between RAIN and RANK was confirmed by co-immunoprecipitation assays using lysates from the RAW clones overexpressing RAIN (FIG. 2A, right panel). This interaction indicates that RAIN may function as an adaptor protein that regulates RANK signal transduction.

### Example 5

RAIN Does Not Inhibit Early Signal Transduction by RANKL

To determine the function of RAIN in RANK signaling, 293 cells were initially co-transfected with various amounts of RAIN in the presence and absence of RANK and determined RAIN's effect on RANK-mediated NF-κB and JNK activation. When expressed in 293 cells, RAIN on its own did not activate NF-κB or JNK. In addition, RAIN modestly suppressed RANK-induced NF-κB reporter activity and did not appear to affect RANK-induced JNK activity (data not shown).

Next, the effect of RAIN's expression on signaling by RANKL was further examined in the RAW clones overexpressing RAIN. RANKL stimulated phosphorylation of ERK both in the parental cells and the RAIN clones (FIG. 2B). Expression of RAIN did not appear to have any effect on RANKL-induced JNK or p38 activation (data not shown). Similarly, RANKL stimulated NF-κB activation in the parental RAW cells and the RAIN clones as determined by gel mobility shift assay, (FIG. 2C, top panel). Additionally, degradation of IκBα, which is required for NF-κB activation, appeared to be similar in the parental RAW cells and RAIN-expressing cells (FIG. 2C, bottom panel). Taken together, these data suggest that expression of RAIN does not interfere with the early signaling cascades initiated by engagement of RANK.

### Example 6

RAIN Stably Expressed in RAW Cells Inhibits RANKL-Induced Osteoclast Formation

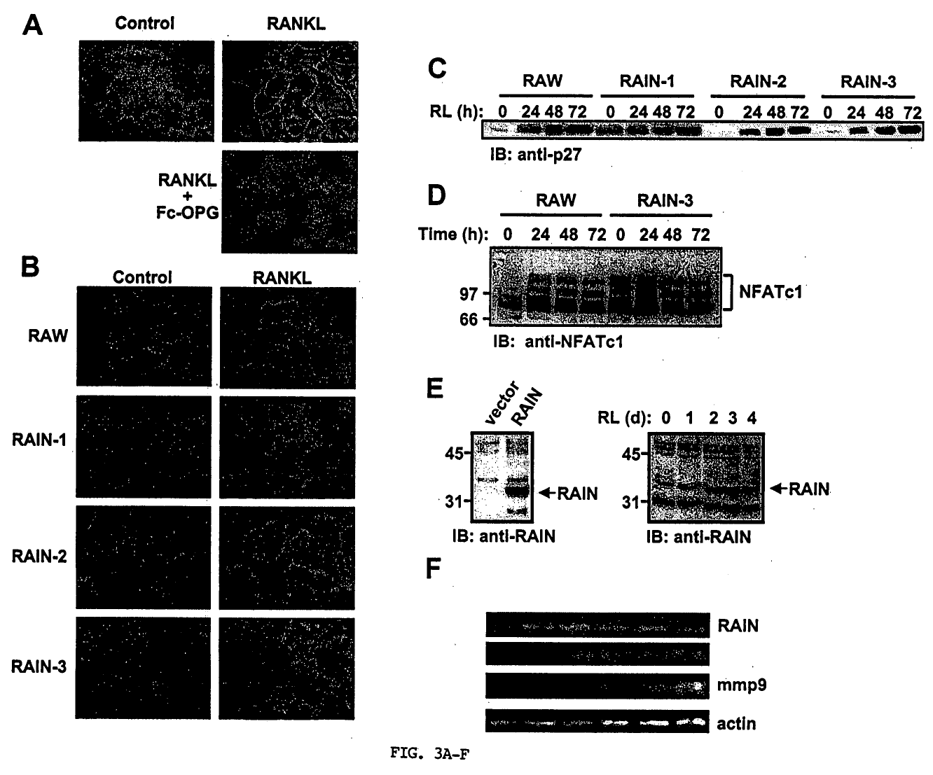

To determine whether RAIN functions in RANKL-mediated osteoclast formation, the ability of RAIN clones to differentiate into tartrate-resistant alkaline phosphatasepositive (TRAP+), multi-nucleated osteoclasts upon RANKL stimulation was examined. RANKL stimulation of RAW cells for 4-5 days causes their differentiation into TRAP+, multi-nucleated osteoclasts, which can be inhibited by co-treatment with Fc-OPG (FIG. 3A). These osteoclasts were functional as they resorbed bone on synthetic bone slides. In contrast, RAW cells stably expressing RAIN did not form multi-nucleated osteoclasts, although they stained positive for TRAP (FIG. 3B). These data suggest that overexpression of RAIN prevents RAW cells from becoming multi-nucleated osteoclasts. Furthermore, RAIN clones plated at various cell densities or stimulated with RANKL for longer time periods failed to form multi-nucleated osteoclasts. Just as the cell cycle inhibitor p27 is upregulated upon RANKL stimulation when RAW cells undergo osteoclast differentiation (Okahashi et al., 2001), so too did RANKL cause p27 upregulation in the RAIN clones, suggesting that RAIN did not interfere with the cell cycle (FIG. 3C).

NFATc1 was shown to be upregulated by RANKL and served as a master switch to cause formation of functional osteoclasts (Ishida et al., 2002; Takayanagi et al., 2002). RANKL induced the expression of NFATc1 in the parental RAW cells and also in RAIN-overexpressing cells, suggesting that this transcription factor was not affected by the expression of RAIN (FIG. 3D). Since the TRAP promoter contains functional NFAT and AP1 binding sites (Takayanagi et al., 2002), these data are consistent with the ability of RANKL to induce TRAP activity in the RAIN-overexpressing cells. Thus, RAW cells overexpressing RAIN did not disrupt RANKL stimulation of early signaling events (i.e., NF-κB, JNK, p38, and ERK) or the induction of p27, TRAP, and NFATc1, but prevented RANKL-mediated osteoclast formation. Taken together, these data imply that RAIN functions at a step during osteoclastogenesis that regulates molecular events associated with formation of the multi-nucleated cell.

### Example 7

RANKL Induces RAIN Expression During Differentiation of RAW Cells Into Osteoclasts

Since RANKL induces the differentiation of RAW cells into functional osteoclasts, RAIN's expression was analyzed during this process. Affinity-purified RAIN antisera was generated that recognized a band with an approximate molecular mass of 34 kDa in 293 cells transfected with RAIN (FIG. 3E, left panel), consistent with the molecular size of FLAG-tagged RAIN ( FIG. 2A). RAIN was expressed weakly in RAW cells, but when treated with RANKL, RAIN is induced starting on day 2 and continuing through day 4 as judged by western blotting with anti-RAIN antibodies (FIG. 3E, right panel). Supporting these data, RANKL stimulated the expression of RAIN mRNA as determined by semi-quantitative RT-PCR (FIG. 3F). That RAIN is expressed at the later stages of RANKL stimulation is consistent with its inability to inhibit early RANKL signaling (FIG. 2) and suggests that it may be involved in other processes of osteoclastogenesis. These data are consistent with the time of differentiation of RAW cells into osteoclasts, in which cellular fusion was observed morphologically as early as 3 days after RANKL stimulation.

### Example 8

RAIN Functions as a Negative Regulator of Osteoclast Formation

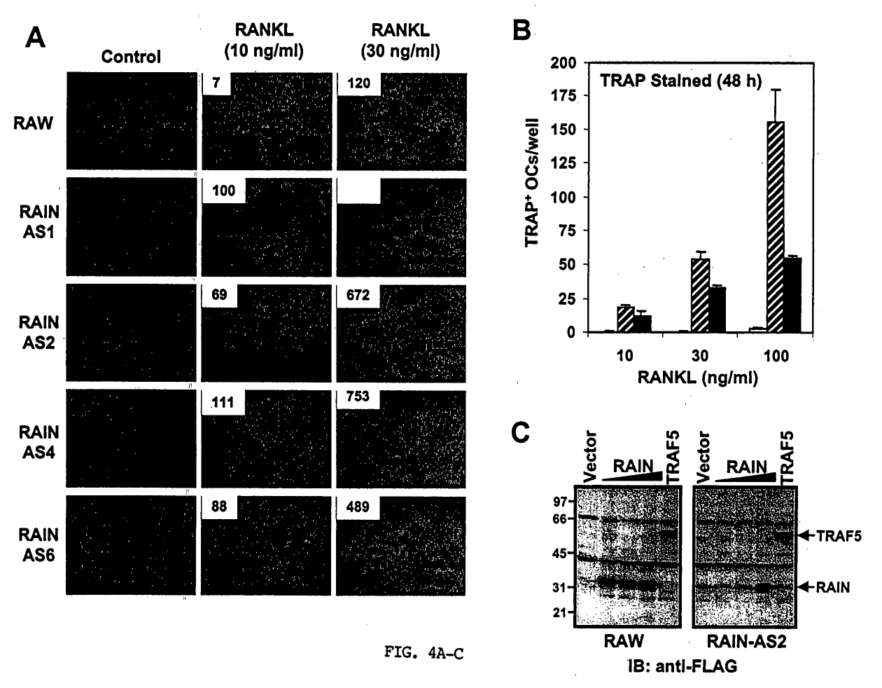

The observations that RAIN interacts with the cytoplasmic domain of RANK without affecting early RANKL-mediated signaling and that RAIN is induced by RANKL led us to hypothesize that RAIN functions in regulating molecular events associated with the fusion of osteoclast progenitors to form multi-nucleated osteoclasts. To test this hypothesis, RAW clones expressing the cDNA of RAIN in the anti-sense orientation were established. Abrogation of RAIN expression caused an increase in the number of multi-nucleated osteoclasts after 72 hr of stimulation with RANKL (FIG. 4A). TRAP+, multi-nucleated osteoclast formation was observed as early as 48 hr after stimulation with RANKL in the RAIN anti-sense clones, and the number of osteoclasts increased with increasing concentrations of RANKL (FIG. 4B). To demonstrate that the RAIN anti-sense indeed inhibited RAIN expression, FLAG-tagged RAIN or TRAF5 was transiently transfected into RAIN anti-sense clones and examined expression by western blotting with anti-FLAG. Expression of FLAG-tagged RAIN, but not FLAG-tagged TRAF5, was suppressed in the RAIN anti-sense clones (FIG. 4C, left panel). However; expression of either FLAG-tagged RAIN or TRAF5 transfected into parental RAW cells was unaffected (FIG. 4C, right panel). Expression of the RAIN anti-sense in these clones did not interfere with any of the early signaling events initiated by RANKL, such as NF-κB, ERK, or JNK activation (data not shown). Furthermore, the RAIN anti-sense clones did not undergo spontaneous osteoclast formation (even when we varied cell density), which suggests that osteoclast formation in these clones was dependent upon RANKL stimulation. Taken together with our previous results that RAIN inhibits osteoclast formation, these data indicate that RAIN functions as a negative regulator specifically at the stage of formation of the multi-nucleated cell.

### Example 9

RAIN Functions in Regulating the Ratio of G- to F-Actin

The reorganization of the actin cytoskeleton has been well established as playing a role in mature osteoclast attachment to bone and bone resorption (Duong et al., 2001; Duong and Rodan, 2000). However, no molecules are known that link RANK stimulation to the formation of the multi-nucleated osteoclast. RAIN may function in osteoclast formation by interacting with molecules associated with cytoskeleton reorganization. Therefore, GST-RAIN pull down assays and co-precipitation experiments were performed, but failed to identify interaction of RAIN with Src, c-Cb1, AKT, Pyk2, WASP, CDC42 and Rho (data not shown).

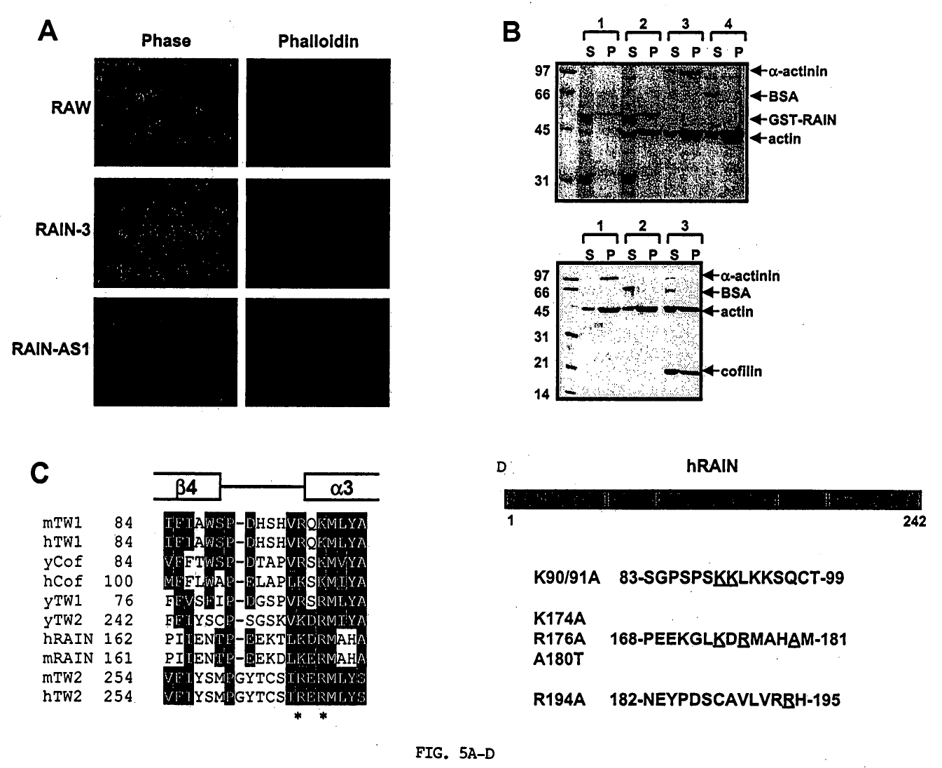

Osteoclasts activated to resorb bone display a distinct organization of F-actin that forms a ring-like cytoskeleton structure (Boyle et al., 2003). The actin ring structure is required for the formation of the sealing zone and the bone resorption function of osteoclasts. When parental RAW cells or RAIN anti-sense cells were treated with RANKL for 5 days and stained with phalloidin-rhodamine an actin-like ring structure is formed, which was absent in cells overexpressing RAIN (FIG. 5A), which is consistent with the absence of multi-nucleated cells.

To dissect the biochemical role of RAIN, the inventor asked whether RAIN interacts with G- or F-actin in co-sedimentation assays (Goode et al., 1998). GST-RAIN appeared predominantly in the supernatant with G-actin and caused a decrease in the formation of F-actin filaments (FIG. 5B, top panel). As controls, alpha-actinin, which is an F-actin-binding protein, was observed only in the pellet, while bovine serum albumin (BSA) was found in the supernatant (FIG. 5B). Cofilin, a protein known to interact with both G- and F-actin and to cause severing of F-actin filaments (Bamburg, 1999), was included in this assay and it also prevented actin polymerization (FIG. 5B, bottom panel). The data suggest that RAIN preferentially interacts with monomeric G-actin and thus may function to prevent its polymerization to filamentous actin.

### Example 10

Point Mutations in RAIN Disrupt its Inhibitory Function

The structures of both cofilin and twinfilin, which are proteins that cause actin depolymerization, have been elucidated by X-ray crystallography (Maciver and Hussey, 2002; Paavilainen et al., 2002). One important feature is the conserved actin-binding domain located on a long α- helix in both twinfilin and cofiln (Paavilainen et al., 2002). When key residues in this actin-binding domain are mutated to alanines, twinfilin loses its ability to interact with monomeric G-actin (Paavilainen et al., 2002). While cofilin binds both G- and F-actin, twinfilin interacts only with G-actin. On close inspection of the amino acid sequence of RAIN, it was observed that a similar actin-binding motif was present in RAIN when aligned with cofilin and twinfilin (FIG. 5C). Of note was the presence of an additional actin-binding motif in RAIN with the consensus, KLKK, which has been suggested to be critical for actin monomer-binding (Paunola et al., 2002) (FIG. 5D).

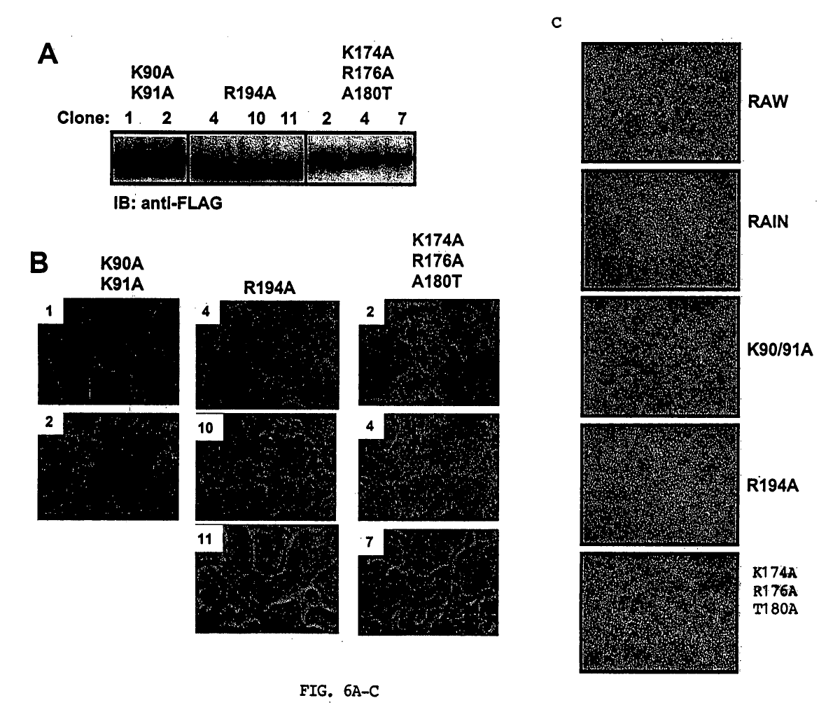

Based upon the potential actin-binding domain in RAIN, a series of point mutations were constructed in RAIN (K90/91A, K174A/R176A/A180T, and R194A) (FIG. 5D). Stable RAW cell lines were generated expressing each of the mutants and selected at least two stable clones of each mutant to examine their effect on osteoclast formation induced by RANKL. All stable RAW clones expressed the RAIN mutant protein to similar levels (FIG. 6A). Unlike the overexpression of wild-type RAIN in RAW cells (FIG. 3B), all clones expressing these mutants differentiated into TRAP+, multi-nucleated osteoclasts (FIG. 6B) and resorbed bone on synthetic bone discs upon RANKL treatment (FIG. 6C). However, these mutants were capable of interacting with endogenous RANK in RAW cells and formed homodimers as indicated by migration during gel filtration chromatography (data not shown), indicating that the mutations did not affect the overall structural integrity of the protein. These data support the hypothesis that RAIN influences actin polymerization, which is required for the formation of the multi-nucleated osteoclast.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,196,265

U.S. Pat. No. 4,554,101

U.S. Pat. No. 4,683,195

U.S. Pat. No. 4,683,202

U.S. Pat. No. 4,800,159

U.S. Pat. No. 4,883,750

U.S. Pat. No. 5,354,855

U.S. Pat. No. 5,582,981

U.S. Pat. No. 5,734,039

U.S. Pat. No. 5,756,291

U.S. Pat. No. 5,780,610

U.S. Pat. No. 5,792,613

U.S. Pat. No. 5,840,867

U.S. Pat. No. 6,004,940

U.S. Pat. No. 6,238,921

U.S. Prov. Appln. 20030077289

Ammann et al., J. Clin. Invest., 99:1699-1703, 1997.

Angel et al., Mol. Cell. Biol., 7:2256, 1987a.

Angel et al., Cell, 49:729, 1987b.

Arch et al., Genes Dev., 12:2821-2830, 1998.

Atchison and Perry, Cell, 46:253, 1986.

Atchison and Perry, Cell, 48:121, 1987.

Ausubel et al., In: Current Protocols in Molecular Biology, John, Wiley & Sons, Inc, New York, 1994.

Baichwal and Sugden, In: Gene Transfer, Kucherlapati (Ed.), NY, Plenum Press, 117-148, 1986.

Bamburg, Annu. Rev. Cell Dev. Biol ., 15:185-230, 1999.

Banedji et al., Cell, 27(2 Pt 1):299-308, 1981.

Banedji et al., Cell, 33(3):729-740, 1983.

Barany and Merrifield, In: The Peptides, Gross and Meienhofer (Eds.), Academic Press, NY, 1-284, 1979.

Baron et al., J. Cell Biol., 101:2210-2222, 1985.

Bellus, J. Macromol. Sci. Pure Appl. Chem., A31(1): 1355-1376, 1994.

Berkhout et al., Cell, 59:273-282, 1989.

Blair and Schlesinger, In: Biology and Physiology of the Osteoclast, Rifkin and Gay (Eds.), CRC Press, Fl, 259-287, 1992.

Blair et al., J. Cell. Biol., 102:1164-1172, 1986.

Blanar et al., EMBO J., 8:1139, 1989.

Bodine and Ley, EMBO J., 6:2997, 1987.

Boshart et al., Cell, 41:521, 1985.

Bosze et al., EMBO J., 5(7):1615-1623, 1986.

Boyle et al., Nature, 423(6937):337-342, 2003.

Braddock et al., Cell, 58:269, 1989.

Brockman et al., Mol. Cell Biol., 15:2809-2818, 1995.

Broder et al., Curr. Biol., 8:1121-1124, 1998.

Bucay et al., Genes Dev., 12:1260-1268, 1998.

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Capaldi et al., Biochem. Biophys. Res. Comm., 74(2):425-433, 1977.

Celander and Haseltine, J. Virology, 61:269, 1987.

Celander et al., J. Virology, 62:1314, 1988.

Chandler et al., Cell, 33:489, 1983.

Chang et al., Mol. Cell. Biol., 9:2153, 1989.

Chatteijee et al., Proc. Natl. Acad. Sci. USA, 86:9114, 1989.

Chen and Okayama, Mol. Cell Biol., 7(8):2745-2752, 1987.

Choi et al., Cell, 53:519, 1988.

Coffin, In: Virology, Fields et al. (Eds.), Raven Press, NY, 1437-1500, 1990.

Cohen et al., J. Cell. Physiol., 5:75, 1987.

Collin-Osdoby, et al., Methods Mol. Med., 80:153-166, 2003.

Costa et al., Mol. Cell. Biol., 8:81, 1988.

Coupar et al., Gene, 68:1-10, 1988.

Cripe et al., EMBO J., 6:3745, 1987.

Culotta and Hamer, Mol. Cell. Biol., 9:1376, 1989.

Dandolo et al., J. Virology, 47:55-64, 1983.

Darnay et al., J. Biol. Chem., 273:20551-2055, 1998.

Darnay et al., J. Biol. Chem., 274:7724-7731, 1999.

De Villiers et al., Nature, 312(5991):242-246, 1984.

Deschamps et al., Science, 230:1174-1177, 1985.

Dougall et al., Genes Dev., 13:2412-2424, 1999.

Duong and Rodan, Cell Motil. Cytoskeleton, 47(3):174-188, 2000.

Duong et al., J. Biol. Chem., 276(10):7484-7492, 2001.

Edbrooke et al., Mol. Cell. Biol., 9:1908, 1989.

Edlund et al., Science, 230:912-916, 1985.

European Appln. 320,308

Fechheimer et al., Proc. Natl. Acad. Sci. USA, 84:8463-8467, 1987.

Feng and Holland, Nature, 334:6178, 1988.

Firak and Subramanian, Mol. Cell. Biol., 6:3667, 1986.

Foecking and Hofstetter, Gene, 45(1):101-105, 1986.

Forster and Symons, Cell, 49(2):211-220, 1987.

Fraley et al., Proc. Natl. Acad. Sci. USA,76:3348-3352, 1979.

Frankel and Pabo, Cell, 55(6):1189-1193, 1988.

Franzoso et al., Genes Dev., 11:3482-3496, 1997.

Freifelder, In: Physical Biochemistry Applications to Biochemistry and Molecular Biology, 2nd ed. Wm. Freeman and Co., NY, 1982.

Friedmann, Science, 244:1275-1281, 1989.

Fujikawa et al., Endocrinol., 137:4058-4060, 1996.

Fujita et al., Cell, 49:357, 1987.

Galibert et al., J. Biol. Chem., 273:34120-34127, 1998.

Gerlach et al., Nature (London), 328:802-805, 1987.

Ghosh and Bachhawat, In: Liver Diseases, Targeted Diagnosis and Therapy Using Specific Receptors and Ligands, Wu et al. (Eds.), Marcel Dekker, NY, 87-104, 1991.

Gilles et al., Cell, 33:717, 1983.

Gloss et al., EMBO J., 6:3735, 1987.

Godbout et al., Mol. Cell. Biol., 8:1169, 1988.

Goding, In: Monoclonal Antibodies: Principles and Practice, 2d ed., Academic Press, Fl., 60-66, and 71-74, 1986.

Gomez-Foix et al., J. Biol. Chem., 267:25129-25134, 1992.

Goodbourn and Maniatis, Proc. Natl. Acad. Sci. USA, 85:1447, 1988.

Goodbourn et al., Cell, 45:601, 1986.

Goode et al., J. Cell Biol., 142(3):723-733, 1998.

Gopal, Mol. Cell. Biol., 5:1188-1190, 1985.

Graham and Prevec, Biotechnology, 20:363-390, 1992.

Graham and Van Der Eb, Virology, 52:456-467, 1973.

Greene et al., Immunology Today, 10:272, 1989

Grosschedl and Baltimore, Cell, 41:885, 1985.

Grunhaus et al., Seminar in Virology, 200(2):535-546, 1992.

Haridas et al., J. Immunol., 160:3152-3162, 1998.

Harland and Weintraub, J. Cell Biol., 101:1094-1099, 1985.

Harlow and Lane, Antibodies: A Laboratory Manual. Cold Spring Harbor Laboratory Press, Cold Spring harbor, N.Y., 553-612, 1988.

Haslinger and Karin, Proc. Natl. Acad. Sci. USA, 82:8572, 1985.

Hauber and Cullen, J. Virology, 62:673, 1988.

Hen et al., Nature, 321:249, 1986.

Hensel et al., Lymphokine Res., 8:347, 1989.

Hermonat and Muzycska, Proc. Natl. Acad. Sci. USA, 81:6466-6470, 1984.

Herr and Clarke, Cell, 45:461, 1986.

Hirochika et al., J. Virol., 61:2599, 1987.

Hirsch et al., Mol. Cell. Biol., 10:1959, 1990.

Hofbauer et al., J. Bone Miner. Res., 15:2-12, 2000.

Holbrook et al., Virology, 157:211, 1987.

Horlick and Benfield, Mol. Cell. Biol., 9:2396, 1989.

Horwich et al. J. Virol., 64:642-650, 1990.

Hsu et al., Proc. Natl. Acad. Sci. USA, 96:3540-3545, 1999.

Hu et al., Genes Dev., 10:2251-2264, 1996.

Huang et al., Cell, 27:245, 1981.

Hug et al., Mol. Cell. Biol., 8:3065, 1988.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Imperiale and Nevins, Mol. Cell. Biol., 4:875, 1984.

Innis et al., PCR Protocols, Academic Press, Inc., San Diego Calif., 1990.

Iotsova et al., Nat. Med., 3:1285-1289, 1997.

Ishida et al., Naturwiss, 89:505-507, 2002

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Johnson et al., Cell, 71:577-586, 1992.

Johnson et al., In: Biotechnology And Pharmacy, Pezzuto et al. (Eds.), Chapman and Hall, N.Y., 1993.

Johnson et al., Mol. Cell. Biol., 9:3393, 1989.

Joyce, Nature, 338:217-244, 1989.

Kadesch and Berg, Mol. Cell. Biol., 6:2593, 1986.

Karin et al., Mol. Cell. Biol., 7:606, 1987.

Karin et al., Mol. Cell. Biol., 7:606, 1987.

Katinka et al., Cell, 20:393, 1980.

Kawamoto et al., Mol. Cell. Biol., 8:267, 1988.

Kiledjian et al., Mol. Cell. Biol., 8:145, 1988.

Kim and Cook, Proc. Natl. Acad. Sci. USA, 84(24):8788-8792, 1987.

Kim et al., FEBS Lett., 443:297-302, 1999.

KIamut et al., Mol. Cell. Biol., 10:193, 1990.

Klein et al., Nature, 327:70-73, 1987.

Koch et al., Mol. Cell. Biol., 9:303, 1989.

Kong et al., Nature, 397:315-323, 1999.

Kriegler and Botchan, In: Eukaryotic Viral Vectors, Gluzman (Ed.), Cold Spring Harbor: Cold Spring Harbor Laboratory, N.Y., 1982.

Kriegler and Botchan, Mol. Cell. Biol., 3:325, 1983.

Kriegler et al., Cell, 38:483, 1984.

Kriegler et al., Cell, 53:45, 1988.

Kuhl et al., Cell, 50:1057, 1987.

Kunz et al., Nucl. Acids Res., 17:1121, 1989.

Kyte and Doolittle, J. Mol. Biol, 157(1):105-132, 1982.

Larsen et al., Proc Natl. Acad. Sci. USA., 83:8283, 1986.

Laspia et al., Cell, 59:283, 1989.

Latimer et al., Mol. Cell. Biol., 10:760, 1990.

Lee et al., Nature, 294:228, 1981.

Lee et al., Nucleic Acids Res., 12:4191-206, 1984.

Levinson et al., Nature, 295:79, 1982.

Levrero et al., Gene, 101: 195-202, 1991.

Lewis et al., Proc. Natl. Acad. Sci. USA, 90:11618-11622, 1993.

Li et al., Proc. Natl. Acad. Sci. USA, 97:1566-1571, 2000.

Lin et al., Mol. Cell. Biol., 10:850, 1990.

Lomaga et al., Genes Dev., 13:1015-1024, 1999.

Ludewig et al., Immunol. Rev., 169:45-54, 1999.

Luria et al., EMBO J., 6:3307, 1987.

Lusky and Botchan, Proc. Natl. Acad. Sci. USA, 83:3609, 1986.

Lusky et al., Mol. Cell. Biol., 3:1108, 1983.

Macejak and Sarnow, Nature, 353:90-94, 1991.

Maciver and Hussey, Genome Biol., 3(5):3007, 2002.

Majors and Varmus, Proc. Natl. Acad. Sci. USA, 80:5866, 1983.

Mann et al., Cell, 33:153-159, 1983.

McNeall et al., Gene, 76:81, 1989.

Merrifield, Science, 232(4748):341-347, 1986.

Michel and Westhof, J. Mol. Biol., 216:585-610, 1990.

Miksicek et al., Cell, 46:203, 1986.

Mizuno et al., Biochem. Biophys. Res. Commun., 247:610-615, 1998.

Mordacq and Linzer, Genes and Dev., 3:760, 1989.

Moreau et al., Nucl. Acids Res., 9:6047, 1981.

Muesing et al., Cell, 48:691, 1987.

Mundy and Roodman, In: Bone and Mineral Research, Peck (Ed.), V:209-280, Elsevier, 1987.

Mundy, Cancer, 80(8 Suppl):1546-56, 1997.

Mundy, Trends Endo. Metab., 1:307-311, 1990.

Ng et al., Nuc. Acids Res., 17:601, 1989.

Nicolas and Rubinstein, In: Vectors: A survey of molecular cloning vectors and their uses, Rodriguez and Denhardt (Eds.), Stoneham: Butterworth, 494-513, 1988.

Nicolau and Sene, Biochim. Biophys. Acta, 721:185-190, 1982.

Nicolau et al., Methods Enzymol., 149:157-176, 1987.

Okahashi et al., J. Cell Biochem., 80(3):339-45, 2001.

Ondek et al., EMBO J., 6:1017, 1987.

Ornitz et al., Mol. Cell. Biol., 7:3466, 1987.

Paavilainen et al., J. Biol. Chem., 277(45):43089-43095, 2002.

Palmiter et al., Nature, 300:611, 1982.

Paskind et al., Virology, 67:242-248, 1975.

Paunola et al., FEBS Lett., 513(1):92-97, 2002.

PCT Appln. WO 90/07641

Pech et al., Mol. Cell. Biol., 9:396, 1989.

Pelletier and Sonenberg, Nature, 334:320-325, 1988.

Perez-Stable and Constantini, Mol. Cell. Biol., 10:1116, 1990.

Picard and Schaffner, Nature, 307:83, 1984.

Pignon et al., Hum. Mutat., 3:126-132, 1994.

Pinkert et al., Genes and Dev., 1:268, 1987.

Poli et al., EMBO J., 13:1189-1196, 1994.

Ponta et al., Proc. Natl. Acad. Sci. USA, 82:1020, 1985.

Porton et al., Mol. Cell. Biol., 10:1076, 1990.

Potter et al., Proc. Natl. Acad. Sci. USA, 81:7161-7165, 1984.

Pullen et al., J. Biol. Chem., 274:14246-14254, 1999.

Queen and Baltimore, Cell, 35:741, 1983.

Quinn et al., Mol. Cell. Biol., 9:4713, 1989.

Redondo et al., Science, 247:1225, 1990.

Reinhold-Hurek and Shub, Nature, 357:173-176, 1992.

Reisman and Rotter, Mol. Cell. Biol., 9:3571, 1989.

Remington's Pharmaceutical Sciences, 15^{th} ed., pages 1035-1038 and 1570-1580, Mack Publishing Company, Easton, Pa., 1980.

Resendez Jr. et al., Mol. Cell. Biol., 8:4579, 1988.

Rickard et al., J. Bone Mineral Res., 11:312-324, 1996.

Ridgeway, In: Vectors: A survey of molecular cloning vectors and their uses, Rodriguez and Denhardt (Eds.), Stoneham:Butterworth, 467-492, 1988.

Ripe et al., Mol. Cell. Biol., 9:2224, 1989.

Rippe et al., Mol. Cell Biol., 10:689-695, 1990.

Rittling et al., Nuc. Acids Res., 17:1619, 1989.

Rojas et al., Proc. West. Pharmacol. Soc., 41:55-56, 1998.

Rosen et al., Cell, 41:813, 1988.

Sakai et al., Genes and Dev., 2:1144, 1988.

Sambrook et al., In: Molecular cloning, Cold Spring Harbor Laboratory Press, Cold Spring Harbor, N.Y., 2001.

Satake et al., J. Virology, 62:970, 1988.

Schaffier et al., J. Mol. Biol., 201:81, 1988.

Scheven et al., Nature, 321:79-81, 1986.

Schwarze et al., Horm Res., 52(4):192-199, 1999.

Searle et al., Mol. Cell. Biol., 5:1480, 1985.

Sharp and Marciniak, Cell, 59:229, 1989.

Shaul and Ben-Levy, EMBO J., 6:1913, 1987.

Sherman et al., Mol. Cell. Biol., 9:50, 1989.

Shevde et al., Proc. Natl. Acad. Sci. USA, 97:7829-7834, 2000.

Singh et al., J. Biol. Chem., 271:31049-31054, 1996.

Sleigh and Lockett, J. EMBO, 4:3831, 1985.

Soriano et al., Cell, 64:693-702, 1991.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Stewart and Young, Solid Phase Peptide Synthesis, 2d. ed., Pierce Chemical Co., 1984.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Takahashi et al., Lab. Invest., 74:827-834, 1996.

Takayanagi et al., Dev. Cell, 3(6):889-901, 2002.

Takebe et al., Mol. Cell. Biol., 8:466, 1988.

Tam et al., J. Am. Chem. Soc., 105:6442, 1983.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (Ed.), NY, Plenum Press, 149-188, 1986.

Thiesen et al., J. Virology, 62:614, 1988.

Treisman, Cell, 42:889, 1985.

Tronche et al., Mol. Biol. Med., 7:173, 1990.

Trudel and Constantini, Genes and Dev. 6:954, 1987.

Tur-Kaspa et al., Mol. Cell Biol., 6:716-718, 1986.

Tyndell et al., Nuc. Acids. Res., 9:6231, 1981.

Vaes, J. Cell Biol., 39:676-697, 1968.

Vannice and Levinson, J. Virology, 62:1305, 1988.

Vasseur et al., Proc. Natl. Acad. Sci. U.S.A., 77:1068, 1980.

Wang and Calame, Cell, 47:241, 1986.

Weber et al., Cell, 36:983, 1984.

Weinberger et al. Mol. Cell. Biol., 8:988, 1984.

Winoto and Baltimore, Cell 59:649, 1989.

Wong et al., Gene, 10:87-94, 1980.

Wong et al., J. Biol. Chem., 273:28355-28359, 1998.

Wong et al., Mol. Cell, 4:1041-1049, 1999.

Wu and Wallace, Genomics, 4:560-569, 1989.

Wu and Wu, Biochemistry, 27:887-892, 1988.

Wu and Wu, J. Biol. Chem., 262:4429-4432, 1987.

Yang et al., Proc. Natl. Acad. Sci. USA, 87:9568-9572, 1990.

Yao et al., J. Biol. Chem., 274:2118-2125, 1999.

Ye et al., Nature, 418(6896):443-337, 2002.

Yutzey et al. Mol. Cell. Biol., 9:1397, 1989.

What is claimed is:

1. An isolated polypeptide comprising at least 10 contiguous amino acids of SEQ ID NO: 2, 4, 5 or 6.

2. The polypeptide of claim 1, comprising at least 15 contiguous amino acids of SEQ ID NO: 2, 4, 5 or 6.

3. The polypeptide of claim 2, comprising at least 20 contiguous amino acids of SEQ ID NO: 2,4,5 or 6.

4. The polypeptide of claim 3, comprising at least 25 contiguous amino acids of SEQ ID NO: 2, 4, 5 or 6.

5. The polypeptide of claim 4, comprising at least 30 contiguous amino acids of SEQ ID NO: 2, 4, 5 or 6.

6. The polypeptide of claim 5, comprising at least 50 contiguous amino acids of SEQ ID NO: 2 or 4.

7. The polypeptide of claim 6, comprising an amino acids sequence of SEQ ID NO: 2, 4, 5 or 6.

8. The polypeptide of claim 1, further comprising a leader sequence operatively coupled to the amino terminus of the polypeptide.

9. The polypeptide of claim 8, wherein the leader signal sequence is a Kaposi fibroblast growth factor signal sequence, HIV-1 Tat (48-60), D-amino acid-substituted HIV-1 Tat (48-60), arginine-substituted HIV-1 Tat (48-60), Drosophila Antennapaedia (43-58), or a polyarginine polypeptide having at least 6 to 8 arginines.

10. An isolated polynucleotide comprising a nucleic acid encoding a RAIN polypeptide.

11. The polynucleotide of claim 10, wherein the nucleic acid sequence is as set forth in SEQ ID NO:1 or SEQ ID NO:2.

12. A method of treating a subject with bone loss comprising inhibiting osteoclast precursor cell fusion by administering a RAIN polypeptide in amount effective to modulate RANK signaling.

13. A method of treating a subject with bone loss comprising inhibiting osteoclast precursor cell fusion by administering an effective amount of an expression vector, wherein the expression vector comprises a polynucleotide encoding a RAIN polypeptide under the transcriptional control of a promoter.

14. The method of claim 13, wherein the promoter is a constitutive promoter.

15. The method of claim 13, wherein the promoter is an inducible promoter.

16. The method of claim 13, wherein the expression vector comprises a viral vector.

17. The method of claim 13, wherein said administration is repeated.

18. The method of claim 16, wherein the viral vector is selected from the group consisting of vaccinia virus, adenovirus, herpesvirus, retrovirus, cytomegalovirus, and adeno-associated virus.

19. The method of claim 13, wherein said expression vector is delivered endoscopically, intraveneously, intraarterially, intramuscularly, intralesionally, percutaneously, or subcutaneously.

20. A method for inhibiting osteoclast precursor cell fusion comprising contacting an osteoclast precursor cell with an expression vector that expresses a RAIN polypeptide.

21. The method of claim 20, wherein the expression vector is a plasmid expression vector.

22. The method of claim 21, wherein the expression vector is a viral expression vector.

23. A method of identifying a modulator of a osteoclast precursor fusion comprising:
(i) providing a cell deficient in a RAIN polypeptide;
(ii) contacting the cell with a candidate substance; and
(iii) comparing osteoclast cell fusion observed when the candidate substance is not added, wherein an alteration in osteoclast cell fusion indicates that the candidate substance is a modulator of a osteoclast cell fusion.

24. The method of claim 23, wherein the candidate substance is a second cell, a cancer cell, a multiple myoloma cell, a peptide, a peptide mimetic or a small molecule.

25. The method of claim 23, wherein the candidate substance is selected from a small molecule library.

26. The method of claim 23, wherein the candidate substance is a protein.

27. The method of claim 23, wherein the candidate substance is a RAIN analogue.

28. The method of claim 23, wherein the cell deficient in a RAIN polypeptide comprises an inactivated RAIN gene.

29. The method of claim 23, wherein the cell deficient in a RAIN polypeptide expresses an antisense RAIN nucleic acid.
